(19) United States
(12) Patent Application Publication
(10) Pub. No.: US 2017/0065631 A1
(43) Pub. Date: Mar. 9, 2017

(54) INHALATION OF NITRIC OXIDE FOR TREATING RESPIRATORY DISEASES

(71) Applicant: Advanced Inhalation Therapies (AIT) Ltd., Beer-yaacov (IL)

(72) Inventors: Yossef Av-Gay, Vancouver (CA); David Greenberg, Omer (IL); Racheli Vizman, Beer-Yaacov (IL)

(73) Assignee: Advanced Inhalation Therapies (AIT) Ltd., Beer-Yaacov (IL)

(21) Appl. No.: 15/272,889

(22) Filed: Sep. 22, 2016

Related U.S. Application Data
(63) Continuation of application No. 14/773,538, filed on Sep. 8, 2015, now abandoned.
(60) Provisional application No. 61/774,038, filed on Mar. 7, 2013.

(51) Int. Cl. A61K 33/00; G01N 33/497; A61B 5/1455; G01N 33/86; G01N 33/68; A61M 16/10; A61K 9/00; G01N 33/84

(52) U.S. Cl. CPC A61K 33/00; A61K 9/007; G01N 33/497; G01N 33/84; G01N 33/86; G01N 33/6863; A61M 16/10; A61B 5/1455; G01N 2033/4975; G01N 2800/12; G01N 2800/52; A61M 2202/0275; A61M 2230/205; A61M 2230/432

(57) ABSTRACT

The present invention relates to a method of treating a human subject suffering from a disease or disorder that is manifested in the respiratory tract or a disease or disorder that can be treated via the respiratory tract, the disease or disorder being associated with a nosocomial infection, the method including subjecting the subject to intermittent inhalation of gNO at a concentration of at least 160 ppm, thereby treating the disease or disorder.

### FIELD AND BACKGROUND OF THE INVENTION

The present invention, in some embodiments thereof, relates to therapy, and more particularly, but not exclusively, to methods and devices for treating respiratory diseases and disorders such as, but not limited to, respiratory diseases or disorders associated with nosocomial infections, and/or with opportunistic infections, by inhalation of gaseous nitric oxide.

Nitric oxide (NO) is a small lipophilic signaling molecule with a small stokes radius and a molecular weight of 30 grams/mol that enables it to cross the glycolipid cell plasma membrane into the cytosol readily and rapidly. NO has an unpaired electron available in its outer orbit that characterizes it as a free radical. NO has been shown to play a critical role in various bodily functions, including the vasodilatation of smooth muscle, neurotransmission, regulation of wound healing and immune responses to infections such as caused by bactericidal action directed toward various organisms. NO has been demonstrated to play an important role in wound healing through vasodilatation, angiogenesis, anti-inflammatory and antimicrobial action.

NO is a common air pollutant and is present in concentrations of 150-650 ppm in cigarette smoke and up to 1200 ppm in cigar and pipe smoke. The National Institute for Occupational Safety and Health (OSHA) and the Environmental Protection Agency have given an inhalation threshold limit value (TLV) as a time-weighted average (TWA) of 25 ppm for NO. The TLV-TWA is the concentration to which a person's respiratory system may be exposed continuously throughout a normal work week without adverse effects and, when represented in ppm hours units, is calculated to be 200 ppm hours. This level is a time-weighted average, that is, the average level of NO should be less than 25 ppm; however, brief exposures to higher concentrations are allowed.

NO is produced by the innate immune response in organs and cells exposed to bacterial and viral infections. These include, among others, the nasopharyngeal airway, lungs and circulating neutrophils and macrophages. NO is also a highly reactive microbicidal free radical that possesses antimicrobial activity against broad range of bacteria, parasites, fungi and viruses. The pore diameter in the cell walls of the microorganisms through which the NO molecule must pass to affect these pathogens is approximately five times wider so that there are few barriers to NO cell penetration. NO is therefore an essential part of the innate immune response. In addition, NO is one of the smallest, yet one of the most important, biological signaling molecules in mammals.

Other than being a well-established direct antimicrobial agent, it has been hypothesized that the antimicrobial and cellular messenger regulatory properties of NO, delivered in an exogenous gaseous form, might easily enter the pulmonary milieu and be useful in optimizing the treatment of uncontrolled pulmonary disease with specific actions directed at reducing bacterial burden, reducing inflammation and improving clinical symptoms.

Some respiratory disorders and physiological conditions can be treated by inhalation of gaseous nitric oxide (gNO). The use of gNO by inhalation can prevent, reverse, or limit the progression of disorders such as acute pulmonary vasoconstriction, traumatic injury, aspiration or inhalation injury, fat embolism in the lung, acidosis, inflammation of the lung, adult respiratory distress syndrome, acute pulmonary edema, acute mountain sickness, post cardiac surgery, acute pulmonary hypertension, persistent pulmonary hypertension of a newborn, perinatal aspiration syndrome, haline membrane disease, acute pulmonary thromboembolism, heparin-protamine reactions, sepsis, asthma and status asthmaticus or hypoxia. Inhaled gNO can also be used to treat cystic fibrosis (CF), chronic pulmonary hypertension, bronchopulmonary dysplasia, chronic pulmonary thromboembolism and idiopathic or primary pulmonary hypertension or chronic hypoxia.

From the toxicological aspect, NO has a half-life in the body of less than 6 seconds and a radius of action of approximately 200 microns from its site of origin, beyond which it is inactivated through binding to sulfhydryl groups of cellular thiols or by nitrosylation of the heme moieties of hemoglobin to form methemoglobin (MetHb). MetHb reductase reduces NO to nitrates in the blood serum. Nitrate has been identified as the predominant nitric oxide metabolite excreted in the urine, accounting for more than 70% of the nitric oxide dose inhaled. Nitrate is cleared from the plasma by the kidney at rates approaching the rate of glomerular filtration. Blood levels of MetHb in healthy humans are typically less than 2%.

Potential side effects of high dose NO treatment hence include the binding of NO to hemoglobin and the formation of MetHb, which could lead to decreased oxygen transport, and the capacity of NO to act as a nitrosylating agent on proteins and other cell constituents. Formation of MetHb and increased levels thereof have been observed in previous studies of gNO inhalation by healthy human individuals, wherein inhalation of gNO at 128 ppm for 3 hours and at 512 ppm for 55 minutes has been reported to drive the levels of MetHb over the safe threshold of 5% [Borgese N. et al., J. Clin. Invest., 1987, 80, 1296-1302; Young J. D. et al., Intensive Care Med., 1994, 20, 581-4 and Young J. D. et al., Brit. J. Anaesthesia, 1996, 76, 652-656].

Thus, concerns have been raised regarding the potential use of NO as a therapeutic agent in various clinical scenarios. To date, studies indicate that acute pulmonary injury, pulmonary edema, hemorrhage, changes in surface tension of surfactant, reduced alveolar numbers and airway responsiveness may be caused by high airway levels of NO, NO₂ and other oxides of nitrogen [Hurford W., Resp. Care, 2005, 50, 1428-9].

Several animal studies conducted in order to evaluate the safety window for gNO exposure were reported on the Primary Medical Review of NDA 20-845 (INOmax nitric oxide gas). Included in these reports is the study referred to as RDR-0087-DS, wherein groups of 10 rats each were exposed to room air or to 80, 200, 300, 400 or 500 ppm gNO for 6 continuous hours per day for up to 7 days. It is reported that all of the animals died on the first day of exposure to 400 and 500 ppm gNO with MetHb levels of 72.5 and 67 percents respectively. Six of the animals treated with 300 ppm gNO died during the first 1-2 days. All deaths were attributed to methemoglobinemia.

In additional studies, rats were exposed continuously to room air, 40, 80, 160, 200 and 250 ppm gNO for 6 hours/day for 28 days. No deaths occurred at gNO concentrations below 200 ppm.

At present, inhalation of gaseous nitric oxide (gNO) as a selective, short acting vasodilator is approved only at 80 ppm for use in full term infants with hypoxic respiratory failure associated with pulmonary hypertension. However, other studies have shown that at such low concentration of inhaled gNO, treatment of adults' respiratory diseases is limited, and the use of higher doses of gNO for treating various medical conditions by inhalation requires in-depth safety studies in humans.

Miller et. al. reported the effect of 1,600 ppm hours gNO against five planktonic (suspended in a liquid) species of methicillin resistant S. aureous (MRSA). An in vitro biofilm MRSA model was also used to compare gNO to the antibiotic vancomycin as an antibacterial agent. For the biofilm experiment, a drip flow reactor was used to grow a MRSA biofilm which was then exposed for eight hours to Ringers lactate, 200 ppm gNO (1,600 ppm hours), air or vancomycin (100-times MIC level). A reduction in the population of all five MRSA planktonic strains was observed after exposure to 1,600 ppm hours of gNO. In the biofilm experiment gNO was also shown to reduce MRSA.

Additional animal studies have shown that gNO at 160-200 ppm can exert potent antimicrobial effects against a broad range of microbes in vitro, ex vivo and in animal models [Kelly T. J. et al., J. Clin. Invest., 1998, 102, 1200-7; McMullin B. et al., Resp. Care., 2005, 50, 1451-6; Ghaffari A. et al., Nitric Oxide, 2005, 12, 129-40; Ghaffari A. et al., Wound Repair Regen., 2007, 15, 368-77; Miller C. C. et al., J. Cutan. Med. Surg. 2004, 8, 233-8; Miller C. C. et al., Nitric Oxide, 2009, 20, 16-23], further suggesting its use as an antimicrobial agent in appropriate concentrations.

Studies conducted in a rat model of Pseudomonas aeruginosa pneumonia tested the antimicrobial effect of a gNO inhaled delivery regimen of intermittent 30 minute exposures of 160-200 ppm gNO, and revealed that 160 ppm gNO in that regiment is effective to reduce the pulmonary bioburden and leukocyte infiltration [Hergott C. A. et al., Am. J. Resp. Crit. Care Med., 2006, 173, A135]. This treatment was also shown to decrease the clinical symptoms of bovine respiratory disease in cattle [Schaefer A. L. et al., Online J. Vet. Res., 2006, 10, 7-16].

Miller, C. C. et al. [J. Cutan. Med. Surg., 2004, 8(4), 233-8] reported on topical treatment of a subject who had a chronic, non-healing wound and presence of a reoccurring biofilm with gNO at a treatment concentration of 200 ppm for two weeks. Within the first three days of treatment, the subject's biofilm was no longer visibly present and at one week, the wound size was reduced by 42%. The subject's ulcer continued to heal following the cessation of nitric oxide exposure.

WO 2005/110441 teaches a method and a corresponding device for combating microbes and infections by delivering intermittent high doses of 160-400 ppm gNO to a mammal for a period of time which cycles between high and low concentration of nitric oxide gas. The regimen involves delivery of 160 ppm gNO for 30 minutes every four hours with 0-20 ppm delivered for the 3.5 hours between the higher concentration deliveries. No experimental data are presented in this publication.

U.S. Pat. No. 7,122,018 teaches topical intermittent exposure to high concentration of nitric oxide ranging 160-400 ppm, for treatment of infected wounds and respiratory infections by a regimen of 4-hour sessions interrupted by 1 hour of rest while monitored methemoglobin blood levels.

U.S. Pat. No. 7,516,742 teaches intermittent high-low dosing by inhalation of gNO to overcome gNO-related toxicity, wherein the high concentration of gNO ranges from 80 to 300 ppm and the low concentration ranges from 0 to 80 ppm, while the regimen may be 160 ppm for 30 minutes every four hours with 20 ppm delivered for the 3.5 hours between the higher concentration deliveries while monitoring the concentration of O₂, NO and NO₂.

U.S. Pat. No. 7,520,866 teaches topical exposure of wounds to gNO at a high concentration ranging 160-400 ppm with a regime of two 4-hour sessions, interrupted by 1 hour of rest, wherein after a first treatment period with high concentration of gNO, a second treatment period at a lower concentration of 5-20 ppm may be provided to restore the balance of nitric oxide and induce collagen expression to aid in the closure of the wound.

U.S. Pat. No. 7,955,294 teaches a method and a corresponding device for topical and inhaled intermittent delivery high-low doses of gNO for a period of time which cycles between high and low concentration, with an exemplary cycle regimen of 160-200 ppm for 30 minutes followed by 0-80 ppm 3.5 hours wherein the cycling regimen can span 1-3 days.

Additional background art includes U.S. Pat. Nos. 8,518,457, 8,083,997, 8,079,998, 8,066,904, 8,057,742, 7,531,133, 7,516,742, 6,432,077, U.S. Patent Application Nos. 2011/0262335, 2011/0259325, 2011/0240019, 2011/0220103 and 2010/0331405, 2011/0112468, 2008/0287861, 2008/0193566, 2007/0116785, 2007/0104653, 2007/0088316, 2007/0086954, 2007/0065473, 2007/0014688, 2006/0207594, 2005/0191372 and WO 2008/095312, WO 2006/071957, WO 2006/110923, WO 2006/110923, WO 2007/057763, WO 2007/057763, WO 2000/30659 and EP 0692984; Miller C. C. et al., Antimicrobial Agents And Chemotherapy, 2007, 51(9), 3364-3366; and Miller C. C. et al., [Resp Care, 2008, 53(11), 1530].

### SUMMARY OF THE INVENTION

The present inventors have studied the effect of intermittent inhalation of gaseous nitric oxide at a concentration of 160 ppm or more by human subjects and have shown that such intermittent inhalation protocol do not result in substantial changes in various physiological parameters of the human subject. Exemplary such parameters are those obtainable on-site in real-time, such as methemoglobin level, end-tidal CO₂ level, and oxygenation, and parameters which are obtainable off-site in the laboratory, such as blood nitrite level, urine nitrite level, and inflammatory markers' level. The present inventors have therefore demonstrated that such a method can be effected safely. Embodiments of the present invention therefore relate to methods of administering gaseous nitric oxide to human subjects in need thereof, while these parameters remain substantially unchanged. The disclosed administration can be used in methods of treating and/or preventing various medical conditions, which are manifested in the respiratory tract, or which can be treated via the respiratory tract, by subjecting a human subject to intermittent inhalation of gaseous nitric oxide at a concentration of 160 ppm or more.

More specifically, embodiments of the present invention relate to the treatment and/or prevention of medical conditions associated with nosocomial infections, and/or with opportunistic infections (e.g., in an immune-compromised subject), and to the treatment and/or prevention of subjects prone to or being at risk to suffer from such conditions.

According to an aspect of some embodiments of the present invention there is provided a method of treating a human subject suffering from a disease or disorder that is manifested in the respiratory tract or a disease or disorder that can be treated via the respiratory tract, the disease or disorder being associated with a nosocomial infection, the method comprising subjecting the subject to intermittent inhalation of gNO at a concentration of at least 160 ppm, thereby treating the disease or disorder.

According to an aspect of some embodiments of the present invention there is provided a method of treating a human subject prone to suffer from, or being at risk of suffering from, a disease or disorder that is manifested in the respiratory tract or a disease or disorder that can be treated via the respiratory tract, the disease or disorder being associated with a nosocomial infection, the method comprising subjecting the subject to intermittent inhalation of gNO at a concentration of at least 160 ppm, thereby treating or preventing the disease or disorder.

According to some embodiments of the present invention, the human subject is prone to suffer the respiratory disease or disorder due to general, environmental and occupational conditions, as described herein.

According to some embodiments of the present invention, the human subject is selected from the group consisting of elderly people, medical staff and personnel (doctors, nurses, caretakers and the likes) of medical facilities and other care-giving homes and long-term facilities, commercial airline crew and personnel (pilots, flight attendants and the likes), livestock farmers and the likes.

According to some embodiments of the present invention, the nosocomial infection is an infection stemming from direct-contact transmission, indirect-contact transmission, droplet transmission, airborne transmission, common vehicle transmission and vector borne transmission.

According to some embodiments of the present invention, the nosocomial infections is caused by an antibiotic resistant bacterium.

According to some embodiments of the present invention, the nosocomial infections is caused by carbapenem-resistant Klebsiella (KPC) or other Enterobacteriaceae, methicillin resistance Staphylococcus aureus (MRSA), Group A Streptococcus, Staphylococcus aureus (methicillin sensitive or resistance), Neisseria meningitides of any serotype and the likes.

According to an aspect of some embodiments of the present invention there is provided a method of treating a human subject suffering from a disease or disorder that is manifested in the respiratory tract or a disease or disorder that can be treated via the respiratory tract, the disease or disorder being an opportunistic infection in an immuno-compromised subject, as described herein, the method comprising subjecting the subject to intermittent inhalation of gNO at a concentration of at least 160 ppm, thereby treating the disease or disorder.

According to an aspect of some embodiments of the present invention there is provided a method of treating a human subject prone to suffer from, or being at risk of suffering from, a disease or disorder that is manifested in the respiratory tract or a disease or disorder that can be treated via the respiratory tract, the disease or disorder being an opportunistic infection in an immuno-compromised subject, as described herein, the method comprising subjecting the subject to intermittent inhalation of gNO at a concentration of at least 160 ppm, thereby treating or preventing the disease or disorder.

According to some embodiments of the present invention, the method further comprises, or is effected while, monitoring, during and following the subjecting, at least one on-site parameter selected from the group consisting of:

a methemoglobin level (SpMet);

an oxygen saturation level (SpO₂);

an end tidal CO₂ level (ETCO₂); and

a fraction of inspired oxygen level (FiO₂),

and/or at least one off-site parameter selected from the group consisting of:

a serum nitrite level (NO₂⁻); and

an inflammatory cytokine plasma level,

in the subject, as these parameters are described herein.

According to some embodiments of the present invention, the method further comprises, or is effected while, monitoring, at least two of the parameters, as described herein.

According to some embodiments of the present invention, the method further comprises, or is effected while, monitoring all of the parameters.

According to some embodiments of the present invention, a change in the at least one of the parameters following the subjecting is less than 2 acceptable deviation units from a baseline, as described herein.

According to some embodiments of the present invention, a change in at least two of the parameters following the subjecting is less than 2 acceptable deviation units from a baseline.

According to some embodiments of the present invention, a change in all of the parameters following the subjecting is less than 2 acceptable deviation units from a baseline.

According to some embodiments of the present invention, a change in at least one of the on-site parameters following the subjecting is less than 2 acceptable deviation units from a baseline.

According to some embodiments of the present invention, a change in at least one of the off-site parameters following the subjecting is less than 2 acceptable deviation units from a baseline.

According to some of any of the embodiments of the present invention, the method further comprises, or is effected while, monitoring urine nitrite level in the subject, as described herein.

According to some embodiments of the present invention, the method further comprises, or is effected while, monitoring a change in the urine nitrite level following the subjecting is less than 2 acceptable deviation units from a baseline.

According to some of any of the embodiments of the present invention, the method further comprises, or is effected while, monitoring in the subject at least one off-site parameter selected from the group consisting of:

a hematological marker;

a vascular endothelial activation factor;

a coagulation parameter;

a serum creatinine level; and

a liver function marker, as these parameters are described herein, in the subject.

According to some embodiments of the present invention, a change in at least one of the off-site parameters following the subjecting is less than 2 acceptable deviation units from a baseline.

According to some of any of the embodiments of the present invention, the method further comprises, or is effected while, monitoring at least one off-site parameter selected from the group consisting of:

a hematological marker;

a vascular endothelial activation factor;

a coagulation parameter;

a serum creatinine level; and

a liver function marker, in the subject, as these parameters are described herein.

According to some embodiments of the present invention, a change in the at least one parameter following the subjecting is less than 2 acceptable deviation units from a baseline.

According to some of any of the embodiments of the present invention, the method further comprises, or is effected while, monitoring in the subject at least one on-site parameter selected from the group consisting of:

a vital sign; and

a pulmonary function, as these parameters are described herein.

According to some embodiments of the present invention, no deterioration is observed in the at least one parameter during and following the subjecting.

According to some of any of the embodiments of the present invention, the intermittent inhalation comprises at least one cycle of continuous inhalation of the gNO for a first time period, followed by inhalation of no gNO for a second time period.

According to some embodiments of the present invention, the first time period is about 30 minutes.

According to some embodiments of the present invention, the second time period ranges from 3 to 5 hours.

According to some embodiments of the present invention, the inhalation comprises from 1 to 6 of the cycles per day.

According to some embodiments of the present invention, the inhalation comprises 5 of the cycles per day.

According to some embodiments of the present invention, during the first time period, the concentration of gNO in the mixture deviates from the concentration of at least 160 ppm by less than 10%.

According to some embodiments of the present invention, during the first time period, a concentration of NO₂ in the mixture is less than 5 ppm.

According to some embodiments of the present invention, during the first time period, a concentration of O₂ in the mixture ranges from 20% to 25%.

According to some embodiments of the present invention, during the first time period, a fraction of inspired oxygen level (FiO₂) in the mixture ranges from 21% to 100%.

According to some embodiments of the present invention, the at least one parameter comprises ETCO₂ and during and following the subjecting, the ETCO₂ is less than 60 mmHg.

According to some embodiments of the present invention, the at least one parameter comprises SpMet and during and following the subjecting, the SpMet is increased by less than 5%.

According to some embodiments of the present invention, the at least one parameter comprises SpO₂ and during the subjecting, a level of the SpO₂ is higher than 89%.

According to some embodiments of the present invention, the at least one parameter comprises serum nitrite/nitrate level and during and following the subjecting, a level of the serum nitrite is less than 2.5/25 micromole per liter respectively.

According to some of any of the embodiments described herein, the intermittent inhalation of gNO is effected during a time period that ranges from 1 to 7 days.

According to some of any of the embodiments described herein, the subjecting is effected by an inhalation device selected from the group consisting of stationary inhalation device, a portable inhaler, a metered-dose inhaler, an atmospherically controlled enclosure and an intubated inhaler.

Unless otherwise defined, all technical and/or scientific terms used herein have the same meaning as commonly understood by one of ordinary skill in the art to which the invention pertains. Although methods and materials similar or equivalent to those described herein can be used in the practice or testing of embodiments of the invention, exemplary methods and/or materials are described below. In case of conflict, the patent specification, including definitions, will control. In addition, the materials, methods, and examples are illustrative only and are not intended to be necessarily limiting.

### BRIEF DESCRIPTION OF THE DRAWINGS

Some embodiments of the invention are herein described, by way of example only, with reference to the accompanying drawings. With specific reference now to the drawings in detail, it is stressed that the particulars shown are by way of example and for purposes of illustrative discussion of embodiments of the invention. In this regard, the description taken with the drawings makes apparent to those skilled in the art how embodiments of the invention may be practiced.

In the drawings:

FIGS. 1A-B present background art bar graphs showing the gNO dosage curve as measured for S. aureus (FIG. 1A) and P. aeruginosa (FIG. 1B) grown on solid media, wherein relative percentage of growth of colony forming units (CFU) at 50, 80, 120 and 160 parts per million (ppm) of gaseous nitric oxide (gNO) compared with growth of CFU in medical air (100%);

FIGS. 2A-C present background art comparative plots showing the viral plaque formation in tissue as a function of time as measured for influenza A/victoria/H3N2 virions after exposure to nitric oxide 160 ppm and 800 ppm continuously for 4 hours (FIG. 2A), the same virions after being exposed to one gNO dose over 30 minute as compared to three 30 minute treatments Q4h (FIG. 2B), and the effect of continuous exposure to gNO at a concentration of 160 ppm for 3 hours of the highly pathogenic Avian Influenza H7N3 (as presented in US 2007/0116785);

FIGS. 3A-D present images showing tissue culture samples harboring human rgRSV30 a common viral lung virus and the causative agent of Broncheolitis, coupled to a green fluorescent protein, and having a starting viral level of 2000 PFU (FIG. 3A), 1000 PFU (FIG. 3B) and 500 PFU (FIG. 3C), upon exposure to 160 ppm gNO for 30 minutes, and a comparative bar plot presenting the plaque reduction in the tested samples to control samples exposed to ambient air;

FIGS. 4A-B present of the data obtained while monitoring methemoglobin (MetHb) levels before, during and after inhalation of 160 ppm of gaseous nitric oxide by 10 healthy human individuals, undergone 5 courses of gNO administration by inhalation daily, each lasting 30 minutes, for 5 consecutive days, while methemoglobin levels were measured using a pulse oximeter, wherein FIG. 4A is a plot of methemoglobin levels by percents as a function of time as measured before (time point 0), during 250 individual 30 minutes gNO administration courses (time interval of 0 to 30 minutes), after the courses (time interval of 30 to 60 minutes) and at 120 minutes, 180 minutes and 240 minutes after gNO administration was discontinued, and FIG. 4B is a plot of methemoglobin levels by percents as a function of time as measured at the beginning and end of 30 minutes gNO administration courses given over the course of 5 days, and followed 8, 12 and 26 days after gNO administration was discontinued;

FIGS. 5A-F present the data obtained while monitoring pulmonary function before, during and after inhalation of 160 ppm of gaseous nitric oxide by 10 healthy human individuals, wherein baseline values of pulmonary function tests were obtained within 7 days prior to gNO administration, and values during gNO administration were obtained on day 2 of the 5-days treatment and other data were obtained after the final gNO administration on day 5 and on days 8, 12 and 26, wherein FIG. 5A presents forced expiratory volume in 1 second (FEV1) in percents (FEV₁), FIG. 5B presents maximum mid-expiratory flow (MMEF), FIG. 5C presents carbon monoxide diffusing capacity (DLCO), FIG. 5D presents forced vital capacity (FVC), FIG. 5E presents total lung capacity (TLC) and FIG. 5F presents residual volume (RV), while all data are presented as means of all ten subjects and absolute differences compared to baseline prior to gNO administration, and statistical differences were assessed by Mann-Whitney test;

FIGS. 6A-F present blood levels of various cytokines before and after inhalation of 160 ppm gaseous nitric oxide by 10 healthy human individuals, as measured from blood samples collected within 7 days prior to gNO administration, each day during the treatment and 8, 12 and 26 days thereafter, wherein FIG. 6A presents the plasma levels of tumor necrosis factor (TNF)α, interleukin (IL)-1β data is presented in FIG. 6B, IL-6 in FIG. 6C, IL-8 in FIG. 6D, IL-10 in FIG. 6E and IL-12p70 in FIG. 6F, as determined by a cytometric bead array while statistical differences are compared by repeated measures ANOVA with Bonferroni post test for parametric data (IL-6, IL-8, IL-10, IL-12p70), or Friedman test with Dunn's post test for non-parametric data (TNF and IL-1b); and

FIGS. 7A-C present plasma levels of angiopoietin (Ang)-1 and Ang-2 before and after inhalation of 160 ppm gaseous nitric oxide by 10 healthy human individuals, as measured in blood sample collected within 7 days prior to gNO inhalation, each day during gNO administration and 8, 12 and 26 days thereafter, wherein plasma levels of Ang 1 are shown in FIG. 7A, Ang-2 in FIG. 7B, and Ang-2/Ang-1 ratios in FIG. 7C, as determined by using a cytometric bead array while statistical differences were assessed compared by Friedman test with Dunn's post test.

### DESCRIPTION OF SPECIFIC EMBODIMENTS OF THE INVENTION

The present invention, in some embodiments thereof, relates to therapy, and more particularly, but not exclusively, to methods and devices for treating respiratory diseases and disorders such as, but not limited to, respiratory diseases or disorders associated with nosocomial infections, and/or with opportunistic infections, by inhalation of gaseous nitric oxide.

The principles and operation of the present invention may be better understood with reference to the figures and accompanying descriptions.

Before explaining at least one embodiment of the invention in detail, it is to be understood that the invention is not limited in its application to the details set forth in the following description or exemplified by the Examples. The invention is capable of other embodiments or of being practiced or carried out in various ways. Also, it is to be understood that the phraseology and terminology employed herein is for the purpose of description and should not be regarded as limiting.

As discussed hereinabove, inhalation of gaseous nitric oxide (gNO) has been shown to be a highly effective broad-spectrum antimicrobial therapy; however, at effective antimicrobial concentration gNO may present serious adverse effects on humans. As shown in previous studies, the currently approved dose of 80 ppm gNO is presumably too low to exert sufficient antimicrobial effects.

As further discussed hereinabove, intermittent dosing and delivery by inhalation of gNO, cycling between high concentrations of gNO for a relatively short period of time and longer periods of no or low concentration of gNO has been suggested for overcoming the problems of NO toxicity. It has been suggested that the high concentration of gNO, delivered according to an intermittent regimen, would be effective in overwhelming the nitric oxide defense mechanisms of pathogens.

It has been further suggested in the art that the high concentration of gNO may be delivered at a concentration of between 80 ppm to 300 ppm, and that the time periods for delivering the high concentration should afford a daily delivery of 600 to 1000 ppm hours.

However, to date, a regimen of intermittent inhalation of gNO, cycling between high concentrations of gNO for a relatively short period of time and longer periods of no or low concentration of gNO has not been applied on humans. Studies demonstrating safety and efficacy of such protocols have never been conducted in human subjects and no protocols were provided for monitoring safety parameters and/or for treating human patients in need of gNO inhalation above the approved dose of 80 ppm.

In the course of devising and practicing novel methods of treating various bacterial, viral and protozoal infections, the present inventors have conducted studies in human subjects, and compiled suitable protocols for safe and effective treatment of a human subject by intermittent inhalation of high concentrations of gNO. The present inventors have demonstrated that short durations of high concentrations of gNO do not cause lung injury or other signs of adverse effects in humans and even improve some vital effects such as lung function and heart rate.

Specifically, the present inventors have conducted a prospective phase I open label safety study in healthy adults, who inhaled 160 ppm gNO for 30 minutes, five times a day, for five consecutive days. Neither significant adverse events nor adverse events attributable to gNO inhalation occurred and all individuals tolerated the gNO treatment courses well. Forced expiratory volume in 1 sec (FEV₁) percentage and other lung function parameters were improved and serum nitrites/nitrates, prothrombin, pro-inflammatory cytokine and chemokine levels, did not differ between baseline and day 5, while methemoglobin levels increased during the study period to a tolerated and accepted level of 0.9%. It was thus demonstrated that inhalation of 160 ppm gNO or more for 30 minutes, about 5 times daily, for 2-7 consecutive days, is safe and well tolerated in healthy individuals.

The present invention, in some embodiments thereof, therefore provides methods of treating human subjects by intermittent inhalation of high concentration of gNO. In some embodiments, the methods disclosed herein are effected while monitoring various parameters relevant for maintaining the desired dosage and regimen, relevant to the safety of the procedure and relevant for efficacy of the treatment.

According to an aspect of some embodiments of the present invention, there is provided a method of treating a human subject in need of inhalation of gaseous NO (gNO), which is effected by subjecting the human subject to intermittent inhalation of gNO at a concentration of at least 160 ppm.

In some embodiments, the method is effected while monitoring various physiological parameters in the subject, as described herein.

According to some embodiments of the invention, subjecting the human subject to gNO intermittent inhalation is effected by intermittently subjecting the human subject to a gaseous mixture which contains gNO at the indicated concentration (a gNO-containing gaseous mixture).

The human subject can be subjected to the inhalation by active or passive means.

By “active means” it is meant that the gaseous mixture is administered or delivered to the respiratory tract of the human subject. This can effected, for example, by means of an inhalation device having a delivery interface adapted for human respiratory organs. For example, the delivery interface can be placed intermittently on the human subject's respiratory organs, whereby when it is removed, the subject breaths ambient air or any other gaseous mixture that is devoid of gNO, as defined herein.

By “passive means” it is meant that the human subject inhales a gaseous mixture containing the indicated dose of gNO without devices for delivering the gaseous mixture to the respiratory tract.

For example, the subject can be subjected to 160 ppm or more gNO in an intermittent regimen by entering and exiting an atmospherically controlled enclosure filled with the gNO-containing mixture of gases discussed herein, or by filling and evacuating an atmospherically controlled enclosure which is in contact with a subject's respiratory tract.

The term “intermittent” is used herein and in the art as an antonym of “continuous”, and means starting and ceasing an action and/or performing an action in intervals.

By “intermittent inhalation” it is meant that the subject is subjected to a gaseous mixture that contains the indicated concentration of gNO intermittently, and thus inhales such a gNO-containing gaseous mixture two or more times with intervals between each inhalation. The subject therefore inhales the gNO-containing gaseous mixture, then stops inhaling a gNO-containing gaseous mixture and inhales instead a gaseous mixture that does not contain the indicated concentration of gNO (e.g., air), then inhales again the gNO-containing gaseous mixture, and so on and so forth.

Hereinthroughout, “a gNO-containing gaseous mixture” is used, for simplicity, to describe a gaseous mixture that contains at least 160 ppm gNO. The gNO-containing mixture can comprise 160 ppm, 170 ppm, 180 ppm, 190 ppm, 200 ppm and even higher concentrations of gNO. Other gaseous mixtures mentioned herein include less than 160 ppm gNO or are being essentially devoid of gNO, as defined herein.

By “essentially devoid of gNO” it is meant no more than 50 ppm, no more than 40 ppm, no more than 30 ppm, no more than 20 ppm, no more than 10 ppm, no more than 5 ppm, no more than 1 ppm and no more than ppb, including absolutely no gNO.

In some embodiments, the method is carried out while maintaining a controlled mixture of inhaled and exhaled gases by standard means for monitoring and controlling, on-site, the contents and/or flow of the mixture to which the subject is subjected to, or that which is delivered through a delivery interface, and/or while monitoring on-site exhaled gases and controlling the intake by feedback in real-time. In some embodiments, the method is effected while monitoring the concentration of gNO, FiO₂/O₂, ETCO₂, and NO₂ in the gaseous mixture to which the subject is exposed or by monitoring other bodily systems non-invasively, such as blood oxygen saturation (SpO₂/SaO₂/DO) and the presence of methemoglobin in the blood (SpMet).

In some embodiments, the concentration of gNO in the gNO-containing gaseous mixture is controlled so as not to deviate from a predetermined concentration by more than 10%. For example, the method is carried out while the concentration of gNO, set to 160 ppm, does not exceed margins of 144 ppm to 176 ppm.

Similarly, the NO₂ content in a gNO-containing gaseous mixture is controlled such that the concentration of NO₂ is maintained lower than 5 ppm.

Further, oxygen level in the gNO-containing gaseous mixture is controlled such that the concentration of O₂ in the mixture ranges from about 20% to about 25%.

Alternatively or in addition, the oxygen level in the gNO-containing gaseous mixture is controlled such that the fraction of inspired oxygen (FiO₂) ranges from about 20% to about 100%.

The phrase “fraction of inspired oxygen” or “FiO₂”, as used herein, refers to the fraction or percentage of oxygen in a given gas sample. For example, ambient air at sea level includes 20.9% oxygen, which is equivalent to FiO₂ of 0.21. Oxygen-enriched air has a higher FiO₂ than 0.21, up to 1.00, which means 100% oxygen. In the context of embodiments of the present invention, FiO₂ is kept under 1 (less than 100% oxygen).

The phrase “end tidal CO₂” or “ETCO₂”, as used herein, refers to the partial pressure or maximal concentration of carbon dioxide (CO₂) at the end of an exhaled breath, which is expressed as a percentage of CO₂ or the pressure unit mmHg. Normal values for humans range from 5% to 6% CO₂, which is equivalent to 35-45 mmHg. Since CO₂ diffuses out of the lungs into the exhaled air, ETCO₂ values reflect cardiac output (CO) and pulmonary blood flow as the gas is transported by the venous system to the right side of the heart and then pumped to the lungs by the right ventricles. A device called capnometer measures the partial pressure or maximal concentration of CO₂ at the end of exhalation. In the context of embodiments of the present invention, a capnometer is used and ETCO₂ levels are monitored so as to afford a warning feedback when ETCO₂ is more than 60 mmHg.

Levels of respiratory NO, NO₂ and O₂ concentration levels (both inhaled and exhaled; inspiratory and expiratory gases) are typically monitored continuously by sampling from a mouthpiece sample port located in an inhalation mask NO, NO₂ and O₂ equipped with an electrochemical analyzer. In the context of embodiments of the present invention, safety considerations requires the absolute minimization of the number of occasions in which NO₂ levels exceed 5 ppm, gNO concentration variations exceeding 10%, and FiO₂/O₂ levels drop below 20% during gNO administration.

According to some embodiments of the present invention, the intermittent inhalation includes one or more cycles, each cycle comprising continuous inhalation of a gaseous mixture containing gNO at the specified high concentration (e.g., at least 160 ppm) for a first time period, followed by inhalation of a gaseous mixture containing no gNO for a second time period. According to some embodiments of the present invention, during the second period of time the subject may inhale ambient air or a controlled mixture of gases which is essentially devoid of gNO, as defined herein.

In some embodiments, the first time period spans from 10 to 45 minutes, or from 20 to 45 minutes, or from 20 to 40 minutes, and according to some embodiments, spans about 30 minutes.

According to some embodiments of the present invention, the second time period ranges from 3 to 5 hours, or from 3 to 4 hours, and according to some embodiments the second time period spans about 3.5 hours.

According to some embodiments of the present invention, this inhalation regimen is repeated 1-6 times over 24 hours, depending on the duration of the first and second time periods.

In some embodiments, a cycle of intermittent delivery of gNO, e.g., 160 ppm for 30 minutes followed by 3.5 hours of breathing no gNO, is repeated from 1 to 6 times a day. According to some embodiments, the cycles are repeated 5 times a day.

According to some embodiments of the present invention, the regimen of 1-5 cycles per day is carried out for 1 to 7 days, or from 2 to 7 days, or from 3 to 7 days. According to some embodiments of the present invention, the intermittent inhalation is effected during a time period of 5 days. However, longer time periods of intermittent gNO administration as described herein, are also contemplated.

In some embodiments, the method is effected while monitoring one or more physiological parameters in the subject and while assuring that no substantial change is effected in the monitored parameters (as demonstrated herein).

In some embodiments, monitoring the one or more physiological parameters is effected by noninvasive measures and/or mild invasive measures.

In some embodiments, monitoring the physiological parameter(s) in the subject is effected by on-site measurement and analysis techniques based on samples collected sporadically, continuously or periodically from the subject on-site in real-time at the subject's bed-side, and/or off-site measurement and analysis techniques based on samples collected sporadically or periodically from the subject which are sent for processing in a off-site which provides the results and analysis at a later point in time.

In the context of some embodiments of the present invention, the phrase “on-site measurement and analysis techniques” or “on-site techniques”, refers to monitoring techniques that inform the practitioner of a given physiological parameter of the subject in real-time, without the need to send the sample or raw data to an off-site facility for analysis. On-site techniques are often noninvasive, however, some rely on sampling from an invasive medical device such as a respiratory tubus, a drainer tube, an intravenous catheter or a subcutaneous port or any other implantable probe. Thus, the phrase “on-site parameters”, as used herein, refers to physiological parameters which are obtainable by online techniques.

Other that the trivial advantage of real-time on-site determination of physiological parameters, expressed mostly in the ability of a practitioner to respond immediately and manually to any critical change thereof, the data resulting from real-time online determination of physiological parameters can be fed into the machinery and be used for real-time feedback controlling of the machinery. In the context of embodiments of the present invention, the term “real-time” also relates to systems that update information and respond thereto substantially at the same rate they receive the information. Such real-time feedback can be used to adhere to the treatment regimen and/or act immediately and automatically in response to any critical deviations from acceptable parameters as a safety measure.

Hence, according to embodiments of the present invention, the term “on-site parameter” refers to physiological and/or mechanical and/or chemical datum which is obtainable and can be put to use or consideration at or near the subject's site (e.g., bed-side) in a relatively short period of time, namely that the time period spanning the steps of sampling, testing, processing and displaying/using the datum is relatively short. An “on-site parameter” can be obtainable, for example, in less than 30 minutes, less than 10 minutes, less than 5 minutes, less than 1 minute, less than 0.5 minutes, less than 20 seconds, less than 10 seconds, less than 5 seconds, or less than 1 second from sampling to use. For example, the time period required to obtain on-site parameters by a technique known as pulse oximetry is almost instantaneous; once the device is in place and set up, data concerning, e.g., oxygen saturation in the periphery of a subject, are available in less than 1 second from sampling to use.

In the context of some embodiments of the present invention, the phrase “off-site measurement and analysis techniques” or “off-site techniques”, refers to techniques that provide information regarding a given physiological parameter of the subject after sending a sample or raw data to an offline, and typically off-site facility, and receiving the analysis offline, sometimes hours or days after the sample had been obtained. Off-site techniques are oftentimes based on samples collected by mild invasive techniques, such as blood extraction for monitoring inflammatory cytokine plasma level, and invasive techniques, such as biopsy, catheters or drainer tubus, however, some off-site techniques rely on noninvasive sampling such as urine and stool chemistry offline and off-site analyses. The phrase “off-site parameters”, as used herein, refers to physiological parameters which are obtainable by off-site laboratory techniques.

Hence, according to embodiments of the present invention, the term “off-site parameter” refers to physiological and/or mechanical and/or chemical datum which is obtain and can be put to use or consideration in a relatively long period of time, namely that the time period spanning the steps of sampling, testing, processing and displaying/using the datum is long compared to on-site parameters. Thus, an “off-site parameter” is obtainable in more than 1 day, more than 12 hours, more than 1 hour, more than 30 minutes, more than 10 minutes, or more than 5 minutes from sampling to use.

An “off-site parameter” is typically obtainable upon subjecting a sample to chemical, biological, mechanical or other procedures, which are typically performed in a laboratory and hence are not performed “on-site”, namely by or near the subject's site.

Noninvasive measures for monitoring various physiological parameters include, without limitation, pulse oximetry, nonintubated respiratory analysis and/or capnometry. Mild invasive measures for monitoring various physiological parameters include, without limitation, blood extraction, continuous blood gas and metabolite analysis, and in some embodiments intubated respiratory analysis and transcutaneous monitoring measures.

The term “pulse oximetry” refers to a noninvasive and on-site technology that measures respiration-related physiological parameters by following light absorption characteristics of hemoglobin through the skin (finger, ear lobe etc.), and on the spectroscopic differences observed in oxygenated and deoxygenated species of hemoglobin, as well as hemoglobin species bound to other molecules, such as carbon monoxide (CO), and methemoglobin wherein the iron in the heme group is in the Fe³⁺ (ferric) state. Physiological parameters that can be determined by pulse oximetry include SpO₂, SpMet and SpCO.

The phrase “nonintubated respiratory analysis”, as used herein, refers to a group of noninvasive and on-site technologies, such as spirometry and capnography, which provide measurements of the physiological pulmonary mechanics and respiratory gaseous chemistry by sampling the inhaled/exhaled airflow or by directing subject's breath to a detector, all without entering the subject's respiratory tract or other orifices nor penetrating the skin at any stage.

The term “spirometry” as used herein, refers to the battery of measurements of respiration-related parameters and pulmonary functions by means of a noninvasive and on-site spirometer. Following are exemplary spirometry parameters which may be used in the context of some embodiments of the present invention:

The spirometric parameter Tidal volume (TV) is the amount of air inhaled and exhaled normally at rest, wherein normal values are based on person's ideal body weight.

The spirometric parameter Total Lung Capacity (TLC) is the maximum volume of air present in the lungs.

The spirometric parameter Vital Capacity (VC) is the maximum amount of air that can expel from the lungs after maximal inhalation, and is equal to the sum of inspiratory reserve volume, tidal volume, and expiratory reserve volume.

The spirometric parameter Slow Vital Capacity (SVC) is the amount of air that is inhaled as deeply as possible and then exhaled completely, which measures how deeply a person can breathe.

The spirometric parameter Forced Vital Capacity (FVC) is the volume of air measured in liters, which can forcibly be blown out after full inspiration, and constitutes the most basic maneuver in spirometry tests.

The spirometric parameter Forced Expiratory Volume in the 1st second (FEV1) is the volume of air that can forcibly be blown out in one second, after full inspiration. Average values for FEV1 in healthy people depend mainly on sex and age, whereas values falling between 80% and 120% of the average value are considered normal. Predicted normal values for FEV1 can be calculated on-site and depend on age, sex, height, weight and ethnicity as well as the research study that they are based on.

The spirometric parameter FEV1/FVC ratio (FEV1%) is the ratio of FEV1 to FVC, which in healthy adults should be approximately 75-80%. The predicted FEV1% is defined as FEV1% of the patient divided by the average FEV1% in the appropriate population for that person.

The spirometric parameter Forced Expiratory Flow (FEF) is the flow (or speed) of air coming out of the lung during the middle portion of a forced expiration. It can be given at discrete times, generally defined by what fraction remains of the forced vital capacity (FVC), namely 25% of FVC (FEF25), 50% of FVC (FEF50) or 75% of FVC (FEF75). It can also be given as a mean of the flow during an interval, also generally delimited by when specific fractions remain of FVC, usually 25-75% (FEF25-75%). Measured values ranging from 50-60% up to 130% of the average are considered normal, while predicted normal values for FEF can be calculated on-site and depend on age, sex, height, weight and ethnicity as well as the research study that they are based on. Recent research suggests that FEF25-75% or FEF25-50% may be a more sensitive parameter than FEV1 in the detection of obstructive small airway disease. However, in the absence of concomitant changes in the standard markers, discrepancies in mid-range expiratory flow may not be specific enough to be useful, and current practice guidelines recommend continuing to use FEV1, VC, and FEV1/VC as indicators of obstructive disease.

The spirometric parameter Negative Inspiratory Force (NIF) is the greatest force that the chest muscles can exert to take in a breath, wherein values indicate the state of the breathing muscles.

The spirometric parameter MMEF or MEF refers to maximal (mid-)expiratory flow and is the peak of expiratory flow as taken from the flow-volume curve and measured in liters per second. MMEF is related to peak expiratory flow (PEF), which is generally measured by a peak flow meter and given in liters per minute.

The spirometric parameter Peak Expiratory Flow (PEF) refers to the maximal flow (or speed) achieved during the maximally forced expiration initiated at full inspiration, measured in liters per minute.

The spirometric parameter diffusing capacity of carbon monoxide (D_{L}CO) refers to the carbon monoxide uptake from a single inspiration in a standard time (usually 10 sec). On-site calculators are available to correct D_{L}CO for hemoglobin levels, anemia, pulmonary hemorrhage and altitude and/or atmospheric pressure where the measurement was taken.

The spirometric parameter Maximum Voluntary Ventilation (MVV) is a measure of the maximum amount of air that can be inhaled and exhaled within one minute. Typically this parameter is determined over a 15 second time period before being extrapolated to a value for one minute expressed as liters/minute. Average values for males and females are 140-180 and 80-120 liters per minute respectively.

The spirometric parameter static lung compliance (Cst) refers to the change in lung volume for any given applied pressure. Static lung compliance is perhaps the most sensitive parameter for the detection of abnormal pulmonary mechanics. Cst is considered normal if it is 60% to 140% of the average value of a commensurable population.

The spirometric parameter Forced Expiratory Time (FET) measures the length of the expiration in seconds.

The spirometric parameter Slow Vital Capacity (SVC) is the maximum volume of air that can be exhaled slowly after slow maximum inhalation.

Static intrinsic positive end-expiratory pressure (static PEEPi) is measured as a plateau airway opening pressure during airway occlusion.

The spirometric parameter Maximum Inspiratory Pressure (MIP) is the value representing the highest level of negative pressure a person can generate on their own during an inhalation, which is expresented by centimeters of water pressure (cmH₂O) and measured with a manometer and serves as n indicator of diaphragm strength and an independent diagnostic parameter.

The term “capnography” refers to a technology for monitoring the concentration or partial pressure of carbon dioxide (CO₂) in the respiratory gases. End-tidal CO₂, or ETCO₂, is the parameter that can be determined by capnography.

Gas detection technology is integrated into many medical and other industrial devices and allows the quantitative determination of the chemical composition of a gaseous sample which flows or otherwise captured therein. In the context of embodiments of the present invention, such chemical determination of gases is part of the on-site, noninvasive battery of tests, controlled and monitored activity of the methods presented herein. Gas detectors, as well as gas mixers and regulators, are used to determine and control parameters such as fraction of inspired oxygen level (FiO₂) and the concentration of nitric oxide in the inhaled gas mixture.

According to some embodiments of the present invention, the measurement of vital signs, such as heart rate, blood pressure, respiratory rate and a body temperature, is regarded as part of a battery of on-site and noninvasive measurements.

The phrase “integrated pulmonary index”, or IPI, refers to a patient's pulmonary index which uses information on inhaled/exhaled gases from capnography and on gases dissolved in the blood from pulse oximetry to provide a single value that describes the patient's respiratory status. IPI, which is obtained by on-site and noninvasive techniques, integrates four major physiological parameters provided by a patient monitor (end-tidal CO₂ and respiratory rate as measured by capnography, and pulse rate and blood oxygenation SpO₂ as measured by pulse oximetry), using this information along with an algorithm to produce the IPI score. IPI provides a simple indication in real time (on-site) of the patient's overall ventilatory status as an integer (score) ranging from 1 to 10. WI score does not replace current patient respiratory parameters, but used to assess the patient's respiratory status quickly so as to determine the need for additional clinical assessment or intervention.

According to some of any of the embodiments described herein, the monitored physiological or chemical parameters include one or more of the following parameters:

a methemoglobin level (SpMet) (an on-line parameter);

an end-tidal CO₂ level (ETCO₂) (an on-line parameter);

an oxygenation level/FIO2 or oxygen saturation level (SpO₂) (an on-line parameter);

an inflammatory cytokine plasma level (an off-line parameter); and

a serum nitrite/nitrate level (NO₂⁻/NO₃⁻) (an off-line parameter);

According to some of any of the embodiments described herein, the monitored physiological or chemical parameters further include one or more of the following parameters:

a urine level of nitrogen dioxide (urine nitrite level) (an off-line parameter);

a vital sign selected from the group consisting of a heart rate, a blood pressure, a respiratory rate and a body temperature (an on-line parameter);

a pulmonary function (spirometric parameter) (an on-line parameter) such as, but not limited to, forced expiratory volume (FEV₁), maximum mid-expiratory flow (MMEF), diffusing capacity of the lung for carbon monoxide (D_{L}CO), forced vital capacity (FVC), total lung capacity (TLC) and residual volume (RV);

a hematological marker (an off-line parameter), such as, but not limited to, a hemoglobin level, a hematocrit ratio, a red blood cell count, a white blood cell count, a white blood cell differential and a platelet count;

a coagulation parameter (an off-line parameter) such as, but not limited to, a prothrombin time (PT), a prothrombin ratio (PR) and an international normalized ratio (INR);

a serum creatinine level (an off-line parameter);

a liver function marker (an off-line parameter) selected from the group consisting of a aspartate aminotransferase (AST) level, a serum glutamic oxaloacetic transaminase (SGOT) level, an alkaline phosphatase level, and a gamma-glutamyl transferase (GGT) level;

a vascular endothelial activation factor (an off-line parameter) selected from the group consisting of Ang-1, Ang-2 and Ang-2/Ang-1 ratio.

Non-limiting examples of inflammatory cytokines include (TNF)α, (IL)-1β, IL-6, IL-8, IL-10 and IL-12p70.

According to some embodiments of the present invention, the method as disclosed herein is such that no substantial change in at least one of the monitored parameters is observed.

In the context of the present embodiments, a change in a parameter is considered substantial when a value of an observation (measurement, test result, reading, calculated result and the likes) or a group of observations falls notably away from a normal level, for example falls about twice the upper limit of a normal level.

A “normal” level of a parameter is referred to herein as baseline values or simply “baseline”. In the context of the present embodiments, the term “baseline” is defined as a range of values which have been determined statistically from a large number of observations and/or measurements which have been collected over years of medical practice with respect to the general human population, a specific sub-set thereof (cohort) or in some cases with respect to a specific person. A baseline is a parameter-specific value which is generally and medically accepted in the art as normal for a subject under certain physical conditions. These baseline or “normal” values, and means of determining these normal values, are known in the art. Alternatively, a baseline value may be determined from or in a specific subject before effecting the method described herein using well known and accepted methods, procedures and technical means. A baseline is therefore associated with a range of tolerated values, or tolerance, which have been determined in conjunction with the measurement of a parameter. In other words, a baseline is a range of acceptable values which limit the range of observations which are considered as “normal”. The width of the baseline, or the difference between the upper and lower limits thereof are referred to as the “baseline range”, the difference from the center of the range is referred to herein as the “acceptable deviation unit” or ADU. For example, a baseline of 4-to-8 has a baseline range of 4 and an acceptable deviation unit of 2.

In the context of the present embodiments, a significant change in an observation pertaining to a given parameter is one that falls more than 2 acceptable deviation unit (2 ADU) from a predetermined acceptable baseline. For example, an observation of 10, pertaining to a baseline of 4-to-8 (characterized by a baseline range of 4, and an acceptable deviation unit of 2), falls one acceptable deviation unit, or 1 AUD from baseline. Alternatively, a change is regarded substantial when it is more than 1.5 ADU, more than 1 ADU or more than 0.5 ADU.

In the context of the present embodiments, a “statistically significant observation” or a “statistically significant deviation from a baseline” is such that it is unlikely to have occurred as a result of a random factor, error or chance.

It is noted that in some parameters or groups of parameters, the significance of a change thereof may be context-dependent, biological system-dependent, medical case-dependent, human subject-dependent, and even measuring machinery-dependent, namely a particular parameter may require or dictate stricter or looser criteria to determine if a reading thereof should be regarded as significant. It is noted herein that in specific cases some parameters may not be measurable due to patient condition, age or other reasons. In such cases the method is effected while monitoring the other parameters.

A deviation from a baseline is therefore defined as a statistically significant change in the value of the parameter as measured during and/or following a full term or a part term of administration the regimen described herein, compared to the corresponding baseline of the parameter. It is noted herein that observations of some parameters may fluctuate for several reasons, and a determination of a significant change therein should take such events into consideration and correct the appropriate baseline accordingly.

Monitoring methemoglobin and serum nitrite levels has been accepted in the art as a required for monitoring the safety of gNO inhalation in a subject. Yet, to date, no clear indication that methemoglobin and serum nitrite levels remain substantially unchanged upon gNO inhalation by a human subject.

According to some embodiments of the present invention, the method comprises monitoring at least one of the parameters described hereinabove.

According to some embodiments, the monitored parameter is methemoglobin level.

As methemoglobin levels can be measured using noninvasive measures, the parameter of percent saturation at the periphery of methemoglobin (SpMet) is used to monitor the stability, safety and effectiveness of the method presented herein. Hence, according to some embodiments of the present invention, the followed parameter is SpMet and during and following the administration, the SpMet level does not exceed 5%, and preferably does not exceed 1%. As demonstrated in the Examples section that follows, a SpMet level of subjects undergoing the method described herein does not exceed 1%.

According to some embodiments, the monitored parameter is serum nitrate/nitrite level.

High nitrite and nitrate levels in a subject's scrum are associated with NO toxicity and therefore serum nitrite/nitrate levels are used to detect adverse effects of the method presented herein. According to some embodiments of the present invention, the tested parameter is serum nitrite/nitrate, which is monitored during and following the treatment and the acceptable level of serum nitrite is less than 2.5 micromole/liter and serum nitrate is less than 25 micromole/liter.

According to some embodiments, the monitored parameter is level of inflammatory markers.

An elevation of inflammatory markers is associated with a phenomenon called “cytokine storm”, which has been observed in subjects undergoing gNO inhalation treatment.

Monitoring inflammatory markers while performing the method as described herein has never been taught heretofore. Moreover, methods involving gNO inhalation at a regimen in which no significant change in inflammatory markers is observed have never been taught heretofore.

According to some embodiments, the method comprises monitoring at least two of the above-mentions parameters.

In some of these embodiments, the monitored parameters are two or all of methemoglobin level, serum nitrite level and inflammatory markers.

While changes in methemoglobin level, serum nitrite level and inflammatory markers are typically observed in subjects subjected to gNO inhalation, the findings that no substantial change in these parameters has been observed in human subjects undergoing the disclosed regimen are surprising.

Hence, according to some embodiments of the present invention, the method as disclosed herein is carried out while monitoring the methemoglobin level (SpMet), the serum nitrite level (NO₂⁻) and a group of inflammatory cytokine plasma level, such as, but not limited to, (TNF)α, (IL)-1β, IL-6, IL-8, IL-10 and IL-12p70 serum levels in the subject, wherein a change in at least one of these parameters is less than 2 acceptable deviation units from a baseline.

According to some of any of the embodiments described herein, the method is effected while monitoring at least one, at least two, or all on-site parameters which include SpMet, SpO₂, and ETCO₂, and/or monitoring at least one or all off-site parameters which include serum nitrite/nitrate level and inflammatory cytokines in the plasma.

For example, the method is effected while monitoring SpMet as an on-site parameter. Alternatively, the method is effected while monitoring SpMet and ETCO₂ as on-site parameters. Alternatively, the method is effected while monitoring SpMet, ETCO₂ and SpO₂ as on-site parameters.

Further alternatively, the method is effected while monitoring SpMet as one on-site parameter, and inflammatory cytokines in the plasma as one off-site parameter. Alternatively, the method is effected while monitoring SpMet and ETCO₂ as on-site parameters, and serum nitrite/nitrate level as one off-site parameter. Alternatively, the method is effected while monitoring SpMet as one on-site parameter, and inflammatory cytokines in the plasma and serum nitrite/nitrate level as off-site parameters. Alternatively, the method is effected while monitoring ETCO₂ as one on-site parameter, and inflammatory cytokines in the plasma and serum nitrite/nitrate level as off-site parameters. Alternatively, the method is effected while monitoring SpO₂ as one on-site parameter, and inflammatory cytokines in the plasma and serum nitrite/nitrate level as off-site parameters.

Further alternatively, the method is effected while monitoring SpMet, ETCO₂ and SpO₂ as on-site parameters, and inflammatory cytokines in the plasma and serum nitrite/nitrate level as off-site parameters.

According to some of any of the embodiments described herein, the method is effected while monitoring at least one, at least two, or all on-site parameters which include SpMet, SpO₂ and ETCO₂, and/or monitoring at least one or all off-site parameters which include serum nitrite/nitrate level and inflammatory cytokines in the plasma, and further monitoring one or more and in any combination of:

a urine NO₂ level (an off-line parameter);

a vital sign (an on-line parameter);

a pulmonary function (an on-line parameter);

a hematological marker (an off-line parameter);

a coagulation parameter (an off-line parameter);

a serum creatinine level (an off-line parameter);

a liver function marker (an off-line parameter);

a vascular endothelial activation factor (an off-line parameter).

According to some of any of the embodiments described herein, the method is effected while monitoring at least one, at least two, or all on-site chemical parameters in the inhaled gas mixture, such as FiO₂ and NO₂.

It is noted herein that for any of the abovementioned embodiments, that the method is effected while no substantial change is observed in any one or more than one or all of the monitored parameters described herein.

According to some embodiments of the present invention, the method is effected while monitoring urine nitrite levels, such that the urine nitrite level is substantially unchanged during and subsequent to carrying out the method as presented herein. It is noted herein that urine nitrite levels may fluctuate for several known reasons, and a determination of a significant change therein should take such events into consideration and correct the appropriate baseline accordingly.

It is noted that urine nitrite level is indicative for the safety of gNO inhalation, yet, has never been monitored heretofore in the context of gNO inhalation in general and in the context of intermittent gNO inhalation as disclosed herein.

According to some embodiments of the present invention, hematological markers, such as the hemoglobin level, the hematocrit ratio, the red blood cell count, the white blood cell count, the white blood cell differential and the platelet count, are substantially unchanged during and subsequent to carrying out the method as presented herein.

According to some embodiments of the present invention, vascular endothelial activation factors, such as Ang-1, Ang-2 and Ang-2/Ang-1 ratio, as well as the serum creatinine level and various liver function markers, such as the aspartate aminotransferase (AST) level, the serum glutamic oxaloacetic transaminase (SGOT) level, the alkaline phosphatase level, and the gamma-glutamyl transferase (GGT) level, are substantially unchanged during and subsequent to carrying out the method as presented herein.

Oxygenation of the subject can be assessed by measuring the subject's saturation of peripheral oxygen (SpO₂). This parameter is an estimation of the oxygen saturation level, and it is typically measured using noninvasive measures, such as a pulse oximeter device. Hence, according to some embodiments of the present invention, the followed parameter during and following the administration is SpO₂, and the level of SpO₂ is higher than about 89%.

According to some embodiments of the present invention, various vital signs, such as the heart rate, the blood pressure, the respiratory rate and the body temperature; and/or various pulmonary functions (spirometric parameter), such as forced expiratory volume (FEV₁), maximum mid-expiratory flow (MMEF), diffusing capacity of the lung for carbon monoxide (D_{L}CO), forced vital capacity (FVC), total lung capacity (TLC) and residual volume (RV); and various coagulation parameters, such as the prothrombin time (PT), the prothrombin ratio (PR) and the international normalized ratio (INR), are substantially unchanged during and subsequent to carrying out the method as presented herein. It is noted that these parameters are regarded as an indication that the general health of the subject is not deteriorating as a result of the medical condition and/or the treatment.

According to some embodiments, the aforementioned general health indicators show an improvement during and subsequent to carrying out the method as presented herein, indicating that the treatment is beneficial to the subject.

Thus, according to some embodiments of the present invention, the method as disclosed herein is effected such that general health indicators as described herein are at least remained unchanged or are improved.

In any one of the embodiments described herein a human subject includes any living human at any age, from neonatals and newborns, to adults and elderly people, at any weight, height, and any other physical state.

According to some embodiments of the present invention, subjecting the subject to intermittent inhalation of gNO at a concentration of at least 160 ppm, as described in any one of the embodiments herein, is used in a method of treating a human subject suffering from, prone to suffer from or being at risk of suffering from, a disease or disorder that is manifested in the respiratory tract or a disease or disorder that can be treated via the respiratory tract, which is associated with a nosocomial infection.

According to an aspect of embodiments of the present invention, there is provided a method for treating a human subject suffering from a disease or disorder that is manifested in the respiratory tract or a disease or disorder that can be treated via the respiratory tract, which is associated with a nosocomial infection, wherein the method is effected by subjecting the subject to intermittent inhalation of gNO at a concentration of at least 160 ppm, essentially as described in any one of the embodiments herein.

According to another aspect of embodiments of the present invention, there is provided a method for treating a human subject prone to suffer from, or being at risk of suffering from, a disease or disorder that is manifested in the respiratory tract or a disease or disorder that can be treated via the respiratory tract, which is associated with a nosocomial infection, wherein the method is effected by subjecting the subject to intermittent inhalation of gNO at a concentration of at least 160 ppm, essentially as described in any one of the embodiments herein.

In the context of embodiments of the present invention “hospital-acquired infection”, also known as a HAI or in medical literature as a “nosocomial infection”, is an infection whose development is more prevalent in a hospital environment, such as one acquired by a patient during a hospitalization or a visit, or one developing among hospital staff. Such infections include fungal and bacterial infections and are aggravated by the reduced resistance of individual patients and the heightened resistance of the pathogens. In the context of the present invention, the term “nosocomial infection” is meant to encompass infections which are more prevalent also in environments other than hospitals and clinics, and include residence facilities for elderly people, veterinary facilities, farms and any livestock-handling facilities, kindergartens and schools, airplanes, boats trains and other mass transportation means and facilities, and any other environment where humans and/or livestock congregate.

By “prone to suffer” in the context of nosocomial infections it is meant that the human subject is at a higher risk of suffering from the indicated disease or disorder compared to a normal subject, such as, but not limited to subjects that spend over than average time (10% and more time than the average ordinary person) in environments wherein nosocomial infections are more prevalent.

According to some embodiments of the present invention, human subjects which are generally more exposed to nosocomial infections and are therefore more prone to suffer from diseases or disorders due to general, environmental and occupational conditions include, without limitation, hospital/clinic patients, elderly people, medical staff and personnel (doctors, nurses, caretakers and the likes) of medical facilities and other care-giving homes and long-term facilities, teachers, train conductors, commercial boat and airline crew and personnel (pilots, flight attendants and the likes), livestock farmers and the likes.

Other incidents and conditions that render a human more susceptible to infections are associated with location, occupation, age, living and environmental conditions, close contact with large groups of people and livestock, close contact with sick people and the likes, all of which are encompassed in the context of the present invention as rendering a human subject prone to suffer from a respiratory disease or disorder associated with nosocomial infection.

According to some embodiments of the present invention, a human subject is in need of preemptive, preventative and prophylactic treatment of a primary and/or secondary disease or disorder as described hereinbelow. Hence, a subject not suffering from any current or manifested disease, and/or a subject that is suspected of being exposed to a pathogen, and/or a subject that suffers from one disease, is treated by any of the methods presented herein in order to prevent the occurrence of another disease or disorder (secondary disease or disorder).

According to some embodiments, the methods presented herein are used to treat or prevent nosocomial infections, such as infections stemming from direct-contact transmission, indirect-contact transmission, droplet transmission, airborne transmission, common vehicle transmission and vector borne transmission.

The methods presented herein are effective to treat diseases and disorders which are caused by any pathogen, as described hereinbelow, including without limitation, pathogens which are known to cause nosocomial infections.

Non-limiting examples of nosocomial infection-causing pathogens include antibiotic resistant bacteria such as carbapenem-resistant Klebsiella (KPC) or other Enterobacteriaceae, Group A Streptococcus species, methicillin resistance Staphylococcus Aureus (MRSA), methicillin sensitive Staphylococcus aureus, E. coli O157:H7, vancomycin-resistant Enterococcus species (VRE), Enterobacter aerogenes, Clostridium difficile, Acinetobacter species such as A. baumannii, Klebsiella pneumonia, Pseudomonas aeruginosa, Neisseria meningitides of any serotype and the likes.

Hence, according to embodiments of the present invention, the methods presented herein can be used to prevent carriage, transmission and infection of pathogenic bacteria and antibiotic resistant pathogenic microorganisms.

According to some embodiments of the present invention, subjecting the subject to intermittent inhalation of gNO at a concentration of at least 160 ppm, as described in any one of the embodiments herein, is used in a method of treating a human subject suffering from, prone to suffer from or being at risk of suffering from, a disease or disorder that is manifested in the respiratory tract or a disease or disorder that can be treated via the respiratory tract, which is associated with an opportunistic infection, e.g., in an immune-compromised subject.

According to an aspect of some embodiments of the present invention, there is provided a method for treating a human subject suffering from a disease or disorder that is manifested in the respiratory tract or a disease or disorder that can be treated via the respiratory tract, wherein the disease or disorder is associated with an opportunistic infection in an immuno-compromised subject.

According to another aspect of embodiments of the present invention, there is provided a method for treating a human subject prone to suffer from, or being at risk of suffering from, a disease or disorder that is manifested in the respiratory tract or a disease or disorder that can be treated via the respiratory tract, wherein the disease or disorder is associated with an opportunistic infection in an immuno-compromised subject.

According to embodiments of the present invention, any of the methods of treating an opportunistic infection in an immuno-compromised subject is effected by subjecting the subject to intermittent inhalation of gNO at a concentration of at least 160 ppm, as described herein.

By “prone to suffer” in the context of opportunistic infections it is meant that the human subject is at a higher risk of suffering from the indicated disease or disorder compared to a normal subject, such as, but not limited to, immune-compromised subjects as described herein.

According to some embodiments, a method of subjecting a human subject to gNO inhalation as described in any one of the embodiments herein, is highly effective for treating respiratory diseases or disorders in subjects which are diagnosed with medical conditions that adversely affect their innate immune system. Humans which are diagnosed with such medical conditions are said to be immuno-compromised or immuno-suppressed.

It is noted herein that immuno-suppression may be a direct result of a pathogen, such as an HIV infection, or an indirect result such as immuno-suppression that occurs in cancer patients being treated with chemotherapeutic agents. Hence, according to some embodiments of the present invention, the methods presented herein are used to treat or prevent a respiratory disease or disorder in immuno-compromised human subject.

Immuno-compromised or immuno-suppressed human subjects are intrinsically more susceptible to opportunistic infections, rendering them prone to suffer from respiratory diseases or disorders. Immuno-suppression may be a result of several conditions, including without limitation, pregnancy, malnutrition, fatigue, recurrent infections, administration of immuno-suppressing agents (such as for organ transplant recipients), advanced HIV infection, chemotherapy (such as for cancer treatment), a genetic predisposition, skin damage, antibiotic treatment, and several other medical procedures.

In some exemplary embodiments, such human subjects include, but are nt limited to, immuno-compromised subjects such as subjects having HIV, cancer patients undergoing or which underwent chemotherapy, and cancer and other patients undergoing or which underwent transplantation, including bone marrow transplantation and transplantation of a solid organ, which are prone to or are at risk to suffer from a respiratory disease or disorder associated with an opportunistic infection.

Alternatively, a human subject in need of gNO treatment is an immuno-compromised subject such as subjects having HIV, cancer patients undergoing or which underwent chemotherapy, cancer and other patients undergoing or which underwent transplantation, including bone marrow transplantation and transplantation of a solid organ, which have been infected or otherwise suffer from a respiratory disease or disorder associated with opportunistic infection.

In the context of embodiments of the present invention, the term “immuno-suppression” is used interchangeably with the term “immunodeficiency” or “immune deficiency”, which is a more general primary or secondary state in which the immune system's ability to fight infectious disease is compromised or entirely absent. While most cases of immunodeficiency are acquired (“secondary”), some subjects are born with defects in their immune system, which is then referred to as primary immunodeficiency.

As used herein, the term “opportunistic infection” refers to bacterial, viral, fungal or protozoan infection caused by opportunistic pathogens that may or may not cause diseases in healthy hosts having a functioning immune system. These pathogens may cause an opportunistic infection since a compromised immune system presents an “opportunity” for such pathogens to thrive in an immuno-compromised subject.

Exemplary opportunistic infections, which occur in human suffering from HIV, and can be treated or prevented by the methods presented herein include, without limitation pneumocystis jiroveci infection, pneumocystis carinii infection and pneumocystis pneumonia (a form of pneumonia caused by the yeast-like fungus).

Other non-limiting examples of opportunistic infection-causing pathogens include Acinetobacter baumanni, Aspergillus sp., Candida albicans, Clostridium difficile, Cryptococcus neoformans, Cryptosporidium, Cytomegalovirus, Geomyces destructans, Histoplasma capsulatum, Isospora belli, Polyomavirus JC polyomavirus (virus that causes Progressive multifocal leukoencephalopathy, Kaposi's Sarcoma caused by Human herpesvirus 8 (HHV8, also called Kaposi's sarcoma-associated herpesvirus KSHV), Legionnaires' Disease (Legionella pneumophila), Microsporidium, Mycobacterium avium complex (MAC) (Nontuberculosis Mycobacterium), Pneumocystis jirovecii (previously known as Pneumocystis carinii f. hominis), Pseudomonas aeruginosa, Staphylococcus aureus, Streptococcus pneumoniae, Streptococcus pyogenes and Toxoplasma gondii.

Exemplary medical conditions which are associated with immunosuppression include AIDS, cancer, primary ciliary dyskinesia (PCD, also known as immotile ciliary syndrome or Kartagener Syndrome).

According to some embodiments of the present invention, any of the methods presented herein is used to treat a human subject suffering from AIDS.

According to some embodiments of the present invention, any of the methods presented herein are used to treat a human subject suffering from cancer.

According to some embodiments of the present invention, any of the methods presented herein can be used to treat or prevent an infection associated with immune deficiency. These include prevention/pre-emptive treatment and treatment of infections in oncology patients.

According to some embodiments of the present invention, any of the methods described herein can be used effectively to treat any respiratory diseases or disorders that occur in humans, as described herein.

According to some of any of the embodiments of the present invention, a human subject in need of gNO inhalation treatment is a human that suffers from a disease or disorder of the respiratory tract.

As used herein, the phrase “respiratory tract” encompasses all organs and tissues that are involved in the process of respiration in a human subject or other mammal subject, including cavities connected to the respiratory tract such as ears and eyes.

A respiratory tract, as used herein, encompasses the upper respiratory tract, including the nose and nasal passages, prenasal sinuses, pharynx, larynx, trachea, bronchi, and nonalveolar bronchioles; and the lower respiratory tract, including the lungs and the respiratory bronchioles, alveolar ducts, alveolar sacs, and alveoli therein.

Respiratory diseases and disorders may be caused by nosocomial infection-causing pathogens or opportunistic infection-cuasing pathogen as a primary pathogen, or can be caused as a secondary condition exacerbated by the primary infection caused by nosocomial/opportunistic infection-causing pathogens.

Respiratory diseases and disorders, primary and/or secondary, which may be caused for any reason and by any pathogen, including, but not limited to, nosocomial infection-causing pathogens, and opportunistic infection-causing pathogen, are treatable by any of the methods presented herein, can be classified as: Inflammatory lung disease; Obstructive lung diseases such as COPD; Restrictive lung diseases; Respiratory tract infections, such as upper/lower respiratory tract infections, and malignant/benign tumors; Pleural cavity diseases; pulmonary vascular diseases; and Neonatal diseases.

According to embodiments of the present invention, restrictive diseases include intrinsic restrictive diseases, such as asbestosis caused by long-term exposure to asbestos dust; radiation fibrosis, usually from the radiation given for cancer treatment; certain drugs such as amiodarone, bleomycin and methotrexate; as a consequence of another disease such as rheumatoid arthritis; hypersensitivity pneumonitis due to an allergic reaction to inhaled particles; acute respiratory distress syndrome (ARDS), a severe lung condition occurring in response to a critical illness or injury; infant respiratory distress syndrome due to a deficiency of surfactant in the lungs of a baby born prematurely; idiopathic pulmonary fibrosis; idiopathic interstitial pneumonia, of which there are several types; sarcoidosis; eosinophilic pneumonia; lymphangioleiomyomatosis; pulmonary Langerhans' cell histiocytosis; pulmonary alveolar proteinosis; interstitial lung diseases (ILD) such as inhaled inorganic substances: silicosis, asbestosis, berylliosis, inhaled organic substances: hypersensitivity pneumonitis, drug induced: antibiotics, chemotherapeutic drugs, antiarrhythmic agents, statins, connective tissue disease: Systemic sclerosis, polymyositis, dermatomyositis, systemic lupus erythematosus, rheumatoid arthritis, infection, atypical pneumonia, pneumocystis pneumonia (PCP), tuberculosis, chlamydia trachomatis, RSV, idiopathic sarcoidosis, idiopathic pulmonary fibrosis, Hamman-Rich syndrome, antisynthetase syndrome, and malignant lymphangitic carcinomatosis; and extrinsic restrictive diseases, such as neuromuscular diseases, including Myasthenia gravis and Guillain barre; nonmuscular diseases of the upper thorax such as kyphosis and chest wall deformities; diseases restricting lower thoracic/abdominal volume due to obesity, diaphragmatic hernia, or the presence of ascites; and pleural thickening.

According to embodiments of the present invention, obstructive diseases include asthma, COPD, chronic bronchitis, emphysema, bronchiectasis, CF, and bronchiolitis.

Respiratory diseases and disorders which are treatable by any of the methods presented herein, can also be classified as acute or chronic; caused by an external factor or an endogenous factor; or as primary or secondary infectious or noninfectious respiratory diseases and disorders.

Diseases and disorders of the respiratory tract include otolaryngological and/or an upper respiratory tract and/or a lower respiratory system diseases and disorders, and are also referred to herein as “respiratory diseases” or “respiratory diseases and disorders”.

Exemplary, and most common, primary or secondary diseases and disorders of the respiratory tract include acute infections, such as, for example, sinusitis, broncholitis, tubercolosis, pneumonia, bronchitis, and influenza, and chronic conditions such as asthma, CF and chronic obstructive pulmonary disease.

According to some embodiments of the present invention, a human subject suitable for the presently disclosed treatment is a human subject that suffers from a primary and/or secondary disease or disorder that is treatable via the respiratory tract.

Since inhaled gNO is absorbed in the lungs, it contacts the blood system and hence can reach other tissues and organs in the biological system. Thus, primary and/or secondary diseases and disorders that are not associated directly to the respiratory tract, yet can be treated by inhalation of agents that show therapeutic effect on such diseases and disorders, can be treated according to embodiments of the present invention. Exemplary such diseases and disorders include, but are not limited to, acidosis, sepsis, leishmaniasis, and various viral infections.

The range of diseases and disorders treatable by any of the methods presented herein spans ophthalmological, otolaryngological and/or an upper respiratory tract and/or a lower respiratory system diseases and disorders, as well as systemic medical conditions.

Exemplary primary and/or secondary diseases and disorders treatable by gNO include, without limitation, a heparin-protamine reaction, a traumatic injury, a traumatic injury to the respiratory tract, acidosis or sepsis, acute mountain sickness, acute pulmonary edema, acute pulmonary hypertension, acute pulmonary thromboembolism, adult respiratory distress syndrome, an acute pulmonary vasoconstriction, aspiration or inhalation injury or poisoning, asthma or status asthmaticus, bronchopulmonary dysplasia, hypoxia or chronic hypoxia, chronic pulmonary hypertension, chronic pulmonary thromboembolism, cystic fibrosis (CF), Aspergilosis, aspergilloma, Cryptococcosis, fat embolism of the lung, haline membrane disease, idiopathic or primary pulmonary hypertension, inflammation of the lung, perinatal aspiration syndrome, persistent pulmonary hypertension of a newborn and post cardiac surgery.

According to some embodiments of the present invention, exemplary treatable primary and/or secondary diseases or disorders include, without limitation, a bacterial-, viral- and/or fungal bronchiolitis, a bacterial-, viral- and/or fungal pharyngitis and/or laryngotracheitis, a bacterial-, viral- and/or fungal pneumonia, a bacterial-, viral- and/or fungal pulmonary infection, a bacterial-, viral- and/or fungal sinusitis, a bacterial-, viral- and/or fungal upper and/or lower respiratory tract infection, a bacterial-, viral- and/or fungal-exacerbated asthma, a respiratory syncytial viral infection, bronchiectasis, bronchitis, chronic obstructive lung disease (COPD), cystic fibrosis (CF), Aspergilosis, aspergilloma, Cryptococcosis, emphysema, otitis, a bacterial-, viral- and/or fungal otitis externa, otitis media, conjunctivitis, uveitis primary ciliary dyskinesia (PCD) and pulmonary aspergillosis (ABPA).

According to some embodiments of the present invention, the primary and/or secondary disease or disorder treatable by gNO is associated with any pathogenic microorganism, including, but not limited to nosocomial infection-causing pathogenic microorganism and/or opportunistic pathogens. The pathogenic microorganisms, according to some embodiments of the present invention, can be, for example, Gram-negative bacteria, Gram-positive bacteria, viruses and viable virions, fungi and parasites.

Exemplary pathogenic microorganisms include, but are not limited to, Acinetobacter baumarmii, Aspergillus niger, Bacteroides vufgatus, Burkhofderia cepacia, Candida albicans, Clostridium perfringes, Enteric Group 137, Enterococcus faecium, Enterohacter aerogenes, Escherichia cofi, Klebsiella pneumoniae, Klebsiella pneumoniae, Klebsiella pneumoniae, Mycobacteria tuberculosis, Pasteurella muftocida, Propbnibacterium acnes, Propbnibacteriumgranulosum, Proteus mirabilis, Providencia rusfigianii, Pseudomonas aeruginosa, Pseudomonas sp., Serratia marcesecens, Staphylococcus aureus, Staphylococcus aureus (FVL positive), Staphylococcus aureus (VNL positive), Staphylococcus aureus MRSA, Staphylococcus aureus MRSA, Staphylococcus aureus MRSA, Streptococci Group B, Streptococci Group D, Streptococci Group G, Streptococcipyrogenes rosenbach Group A, Streptococcus pneumoniae, Trichophyton meriagrophytes, Trichophyton rubrum, and Vibrio vuMucus.

Exemplary Gram-negative bacteria include, but are not limited to, Proteobacteria, Enterobacteriaceae, Acinetobacter baumannii, Bdellovibrio, Cyanobacteria, Enterobacter cloacae, Escherichia coli, Helicobacter, Helicobacter pylori, Hemophilus influenza, Klebsiella pneumonia, Legionella, Legionella pneumophila, Moraxella, Moraxella catarrhalis, Neisseria gonorrhoeae, Neisseria meningitides, Proteus mirabilis, Pseudomonas, Pseudomonas aeruginosa, Salmonella, Salmonella enteritidis, Salmonella typhi, Serratia marcescens, Shigella, Spirochaetes and Stenotrophomonas.

Exemplary Gram-positive bacteria include, but are not limited to, Bacillus species such as B. alcalophilus, B. alvei, B. aminovorans, B. amyloliquefaciens, B. aneurinolyticus, B. anthracis, B. aquaemaris, B. atrophaeus, B. boroniphilus, B. brevis, B. caldolyticus, B. centrosporus, B. cereus, B. circulans, B. coagulans, B. firmus, B. flavothermus, B. fusiformis, B. globigii, B. infernus, B. larvae, B. laterosporus, B. lentus, B. licheniformis, B. megaterium, B. mesentericus, B. mucilaginosus, B. mycoides, B. natto, B. pantothenticus, B. polymyxa, B. pseudoanthracis, B. pumilus, B. schlegelii, B. sphaericus, B. sporothermodurans, B. stearothermophilus, B. subtilis, B. thermoglucosidasius, B. thuringiensis, B. vulgatis and B. weihenstephanensis, Clostridium species such as C. acetobutylicum, C. aerotolerans, C. argentinense, C. baratii, C. beijerinckii, C. bifermentans, C. botulinum, C. butyricum, C. cadaveris, C. cellulolyticum, C. chauvoei, C. clostridioforme, C. colicanis, C. difficile, C. estertheticum, C. fallax, C. feseri, C. formicaceticum, C. histolyticum, C. innocuum, C. kluyveri, C. lavalense, C. ljungdahlii, C. novyi, C. oedematiens, C. paraputrificum, C. perfringens, C. phytofermentans, C. piliforme, C. ragsdalei, C. ramosum, C. scatologenes, C. septicum, C. sordellii, C. sporogenes, C. sticklandii, C. tertium, C. tetani, C. thermocellum, C. thermosaccharolyticum, C. tyrobutyricum, Corynebacterium species such as C. accolens, C. afermentans, C. amycolatum, C. aquaticum, C. argentoratense, C. auris, C. bovis, C. diphtheriae, C. equi, C. flavescens, C. glucuronolyticum, C. glutamicum, C. granulosum, C. haemolyticum, C. halofytica, C. jeikeium, C. macginleyi, C. matruchotii, C. minutissimum, C. parvum, C. propinquum, C. pseudodiphtheriticum, C. pseudotuberculosis, C. pyogenes, C. renale, C. spec, C. striatum, C. tenuis, C. ulcerans, C. urealyticum, C. urealyticum and C. xerosis, Listeriai species such as L. grayi, L. innocua, L. ivanovii, L. monocytogenes, L. murrayi, L. seeligeri and L. welshimeri, Staphylococcus species such as S. arlettae, S. aureus, S. auricularis, S. capitis, S. caprae, S. carnosus, S. chromogenes, S. cohnii, S. condimenti, S. delphini, S. devriesei, S. epidermidis, S. equorum, S. felis, S. fleurettii, S. gallinarum, S. haemolyticus, S. hominis, S. hyicus, S. intermedius, S. kloosii, S. leei, S. lentus, S. lugdunensis, S. lutrae, S. massiliensis, S. microti, S. muscae, S. nepalensis, S. pasteuri, S. pettenkoferi, S. piscifermentans, S. pseudintermedius, S. pseudolugdunensis, S. pulvereri, S. rostri, S. saccharolyticus, S. saprophyticus, S. schleiferi, S. sciuri, S. simiae, S. simulans, S. stepanovicii, S. succinus, S. vitulinus, S. warneri and S. xylosus, and Streptococcus species such as S. agalactiae, S. anginosus, S. bovis, S. canis, S. constellatus, S. dysgalactiae, S. equinus, S. iniae, S. intermedius, S. initis, S. mutans, S. oralis, S. parasanguinis, S. peroris, S. pneumoniae, S. pyogenes, S. ratti, S. salivarius, S. sanguinis, S. sobrinus, S. suis, S. thermophilus, S. uberis, S. vestibularis, S. viridians and S. zooepidemicus.

As discussed hereinabove, the primary and/or secondary disease or disorder which can be treated by effecting the method presented herein to a human subject suffering from a disease or disorder that is associated with a nosocomial infection or with an opportunistic infection, includes bacterial-, viral- and/or fungal bronchiolitis, bacterial-, viral- and/or fungal pharyngitis and/or laryngotracheitis, bacterial-, viral- and/or fungal sinusitis, bacterial-, viral- and/or fungal upper and/or lower respiratory tract infection, bacterial-, viral- and/or fungal-exacerbated asthma, bacterial-, viral-, fungal- and/or parasitic pneumonia, the common cold, cystic fibrosis related infections, aspergillosis, aspergilloma, respiratory syncytial viral infections, acidosis or sepsis, oral fungal infections, bronchitis, candidiasis of the oral cavity (thrush), canker sores, epiglottitis (supraglottitis), halitosis, herpes, laryngitis, laryngotracheitis, nasopharyngitis, otitis externa and otitis media, conjunctivitis, uveitis (and other eye infections) pharyngitis, pulmonary aspergillosis (ABPA), respiratory syncytial virus infections, rhinitis, rhinopharyingitis, rhinosinusitis, stomatitis, tonsillitis, tracheitis, tuberculosis, cryptococcosis and tympanitis.

In general, any of the methods presented herein are suitable for treating a human subject suffering from any primary and/or secondary disease or a disorder which is associated, directly or indirectly, with a pathogenic microorganism, as described herein. The methods are effected by subjecting the subject to intermittent inhalation regimen of gNO at a concentration of at least 160 ppm, as described in any of the present embodiments.

In general, any of the methods presented herein are suitable for treating a human subject suffering from any primary and/or secondary disease or disorder that is manifested in the respiratory tract or a disease or disorder that can be treated via the respiratory tract, which are effected by subjecting the subject to intermittent inhalation regimen, gNO at a concentration of at least 160 ppm, as described in any of the present embodiments.

In general, any of the methods presented herein are suitable for treating a human subject prone to suffer from any primary and/or secondary disease or disorder that is manifested in the respiratory tract or a disease or disorder that can be treated via the respiratory tract, as described herein, which are effected by subjecting the subject to intermittent inhalation regimen, gNO at a concentration of at least 160 ppm, as described in any of the present embodiments. Such a method can be regarded as a preventive or prophylaxis treatment of the subject.

In general, any of the methods presented herein are suitable for treating a human subject suffering from a primary and/or secondary ophthalmological, otolaryngological and/or upper respiratory tract disease or disorder, as described herein, which are effected by subjecting the subject to intermittent inhalation regimen, gNO at a concentration of at least 160 ppm, as described in any of the present embodiments.

According to some embodiments of the present invention, the otolaryngological and/or upper respiratory tract disease and disorder involves an infection or an inflammation of a bodily site selected from the group consisting of an ear cavity, a nasal cavity, a sinus cavity, an oral cavity, a pharynx, a epiglottis, a vocal cord, a trachea, an apex and an upper esophagus.

According to some embodiments of the present invention, the ophthalmological, otolaryngological and/or upper respiratory tract diseases and disorders include, without limitation, the common cold, a stomatognathic disease, amigdalitis, an oral fungal infection, bacterial-, viral- and/or fungal sinusitis, bronchitis, candidiasis of the oral cavity (thrush), canker sores, epiglottitis (supraglottitis), halitosis, herpes, laryngitis, laryngotracheitis, nasopharyngitis, otitis (externa and media), conjunctivitis, uveitis and other eye infections, pharyngitis, rhinitis, rhinopharyingitis, rhinosinusitis, stomatitis, tonsillitis, tracheitis, tracheitis and tympanitis.

In general, any of the methods presented herein are suitable for treating a human subject suffering from a primary and/or secondary disease or disorder of the lower respiratory system, as described herein, essentially by intermittent inhalation regimen, gNO at a concentration of at least 160 ppm, as described in any of the embodiments herein.

According to some embodiments of the present invention, diseases and disorders of the lower respiratory system include, without limitation, an obstructive condition, a restrictive condition, a vascular disease and an infection, an inflammation due to inhalation of foreign matter and an inhaled particle poisoning.

According to some embodiments of the present invention, the obstructive condition includes, without limitation, a chronic obstructive lung disease (COPD), emphysema, bronchiolitis, bronchitis, asthma and viral, bacterial and fungal exacerbated asthma; the restrictive condition includes, without limitation, fibrosis, cystic fibrosis, sarcoidosis, alveolar damage and pleural effusion; the vascular disease includes, without limitation, pulmonary edema, pulmonary embolism and pulmonary hypertension; the infection includes, without limitation, respiratory syncytial virus infection, tuberculosis, a viral-, bacterial-, fungal-, and/or parasitic pneumonia, idiopathic pneumonia; and the inflammation due to inhalation of foreign matter and an inhaled particle poisoning includes, without limitation, smoke inhalation, asbestosis and exposure to particulate pollutants and fumes.

According to some embodiments of the present invention, any of the methods of treating or preventing a subject as described herein encompasses all of the conditions, disease and disorders described hereinabove for subjects which can be treated by gNO inhalation.

The methods presented herein are fast and effective in treating a resent medical condition, disease or disorder. Moreover, the methods presented herein are effective in preventing a primary and/or secondary disease or disorder from taking hold in a subject which is prone to suffer from, contract or develop a disease or disorder which is associated with the respiratory tract. According to some embodiments, some methods of gNO inhalation are particularly useful in preventing a disease or disorder, while other methods are particularly effective in treating an existing primary and/or secondary disease or disorder.

According to some embodiments of the present invention, any of the methods of treatment presented herein further includes monitoring, during and following administration gNO, one or more of the parameters as described in any of the embodiments hereinabove.

In some embodiments, the methods are effected while monitoring one, two, etc., or all of:

a methemoglobin level (SpMet) (an on-line parameter);

an end-tidal CO₂ level (ETCO₂) (an on-line parameter);

an oxygenation level or oxygen saturation level (SpO₂) (an on-line parameter);

an inflammatory cytokine plasma level (an off-line parameter); and

a serum nitrite/nitrate level (NO₂⁻/NO₃⁻) (an off-line parameter).

In some embodiments, no significant deviation from baseline, as described herein, is shown in at least one, two, three, four or all of the above parameters, when monitored, as described herein

Other parameters and markers may be monitored as well, as presented hereinabove, while showing significant deviation from a baseline, and various general health indicators show no change to the worse, or an improvement, as presented hereinabove.

According to some embodiments of the present invention, in any of the methods of treatment presented herein, the gNO administration can be effected by an inhalation device which includes, without limitation, a stationary inhalation device, a portable inhaler, a metered-dose inhaler and an intubated inhaler.

An inhaler, according to some embodiments of the present invention, can generate spirometry data and adjust the treatment accordingly over time as provided, for example, in U.S. Pat. No. 5,724,986 and WO 2005/046426. The inhaler can modulate the subject's inhalation waveform to target specific lung sites. According to some embodiments of the present invention, a portable inhaler can deliver both rescue and maintenance doses of gNO at subject's selection or automatically according to a specified regimen.

According to some embodiments of the present invention, an exemplary inhalation device may include a delivery interface adaptable for inhalation by a human subject.

According to some embodiments of the present invention, the delivery interface includes a mask or a mouthpiece for delivery of the mixture of gases containing gNO to a respiratory organ of the subject.

According to some embodiments of the present invention, the inhalation device further includes a gNO analyzer positioned in proximity to the delivery interface for measuring the concentration of gNO, oxygen and nitrogen dioxide flowing to the delivery interface, wherein the analyzer is in communication with the controller.

According to some embodiments of the present invention, subjecting the subject to the method described herein is carried out by use of an inhalation device which can be any device which can deliver the mixture of gases containing gNO to a respiratory organ of the subject. An inhalation device, according to some embodiments of the present invention, includes, without limitation, a stationary inhalation device comprising tanks, gauges, tubing, a mask, controllers, values and the likes; a portable inhaler (inclusive of the aforementioned components), a metered-dose inhaler, a an atmospherically controlled enclosure, a respiration machine/system and an intubated inhalation/respiration machine/system. An atmospherically controlled enclosure includes, without limitation, a head enclosure (bubble), a full body enclosure or a room, wherein the atmosphere filling the enclosure can be controlled by flow, by a continuous or intermittent content exchange or any other form of controlling the gaseous mixture content thereof.

It is expected that during the life of a patent maturing from this application many relevant medical procedures involving inhalation of gNO will be developed and the scope of the term treatment by inhalation of gNO is intended to include all such new technologies a priori.

As used herein the term “about” refers to ±10%.

The terms “comprises”, “comprising”, “includes”, “including”, “having” and their conjugates mean “including but not limited to”.

The term “consisting of” means “including and limited to”.

The term “consisting essentially of” means that the composition, method or structure may include additional ingredients, steps and/or parts, but only if the additional ingredients, steps and/or parts do not materially alter the basic and novel characteristics of the claimed composition, method or structure.

As used herein, the singular form “a”, “an” and “the” include plural references unless the context clearly dictates otherwise. For example, the term “a compound” or “at least one compound” may include a plurality of compounds, including mixtures thereof.

Throughout this application, various embodiments of this invention may be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals therebetween.

As used herein the term “method” refers to manners, means, techniques and procedures for accomplishing a given task including, but not limited to, those manners, means, techniques and procedures either known to, or readily developed from known manners, means, techniques and procedures by practitioners of the chemical, pharmacological, biological, biochemical and medical arts.

As used herein, the term “treating” includes abrogating, substantially inhibiting, slowing or reversing the progression of a condition, substantially ameliorating clinical or aesthetical symptoms of a condition, and substantially preventing the appearance of clinical or aesthetical symptoms of a condition, namely preemptive, preventative and prophylactic treatment.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination or as suitable in any other described embodiment of the invention. Certain features described in the context of various embodiments are not to be considered essential features of those embodiments, unless the embodiment is inoperative without those elements.

Various embodiments and aspects of the present invention as delineated hereinabove and as claimed in the claims section below find experimental or calculated support in the following examples.

### EXAMPLES

Reference is now made to the following examples, which together with the above descriptions illustrate some embodiments of the invention in a non limiting fashion.

### Example 1

### Background Art

### Determination of Effective Antimicrobial Level of gNO

The direct effect of gNO on bacteria was studied by determining the concentration of gNO which is lethal for microbes. Once an optimal dose was estimated, timing study was conducted to optimize the duration of exposure of the microbes to gNO.

For these initial studies, highly dense inoculums of P. aeruginosa and S. aureus suspensions (10⁸ chum) were plated onto agar plates. These plates were then exposed to various concentrations of gNO in an exposure device in order to evaluate the effect on colony growth.

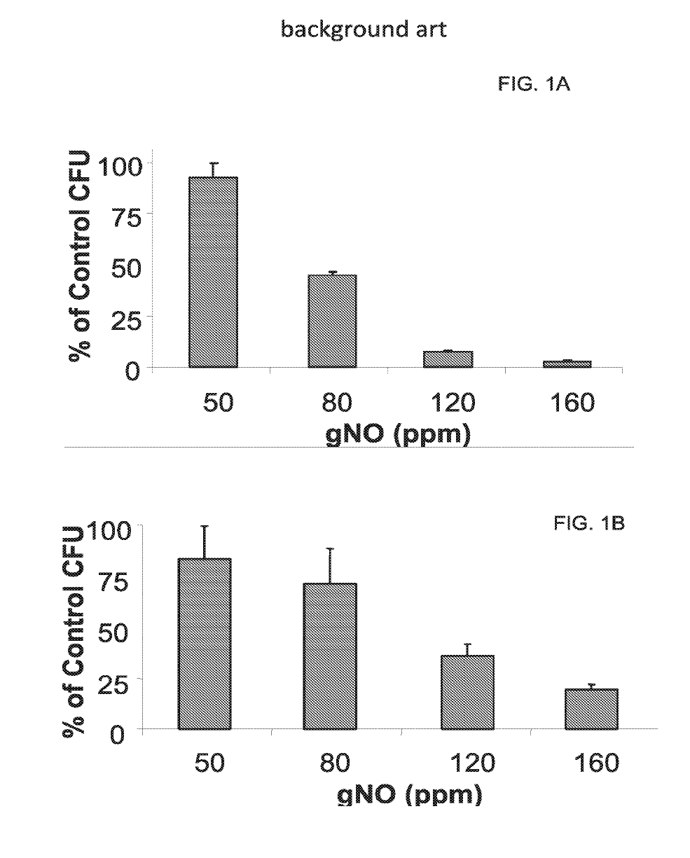

FIGS. 1A-B present bar-plot showing the gNO dosage curve on as measured for S. aureus (FIG. 1A) and P. aeruginosa (FIG. 1B) grown on solid media, wherein relative percentage of growth of colony forming units (CFU) at 50, 80, 120 and 160 parts per million (ppm) of gaseous nitric oxide (gNO) compared with growth of CFU in medical air (100%).

As can be seen in FIGS. 1A-B, the results confirmed that gNO has an inhibitory effect on P. aeruginosa and S. aureus growth. The data provided preliminary evidence that there was a time and dose relationship trend, with the amount of bactericidal (antibiotic) activity increasing with increased time of exposure and concentration of gNO. As the concentration of gNO increased, the number of colonies growing on the plates decreased. Although there was a downward bactericidal trend towards 5-10% survival, none of the data showed a 100% bactericidal effect. Some bacteria may have survived because the materials and chemicals in the agar may have reacted with the gNO and buffered the effect.

It is noted that bacterial colonies remained the same in size and number after being transferred to a conventional incubator for 24 hours, whereas controls increased in number and size to the degree that they could not be counted. This observation suggested that gNO exposure prevented the growth of the bacteria, and may have killed the bacteria at some point during the gNO exposure.

These results demonstrated that gNO had a bacteristatic effect on both bacterial strains, and as a result, subsequent studies were designed to further study the bactericidal effects of gNO. The studies demonstrated that levels of gNO greater than 120 ppm reduced the colony formation by greater than 90%. Studies then followed indicating that the time required to achieve this effect occurred between 8-12 hours.

A similar procedure was used to determine the time required to induce an effective bactericidal effect with 200 parts per million gNO, a concentration just above the dose used in the dose-ranging study presented hereinabove, on a representative collection of drug resistant gram-positive and gram-negative strains of bacteria associated with clinical infection.

A successful bactericidal effect was defined as a decrease in bacteria greater than 3 log₁₀ CFU/mL. Further, C. albicans, Methicillin Resistant S. aureus (MRSA), a particularly resistant strain of P. aeruginosa from a cystic fibrosis patient, Group B Streptococcus, and M. smegmatis were also included to evaluate if yeasts, a multi-drug resistant strain of bacteria and actinomycetes have a similar response. These bacteria represent a comprehensive variety of drug resistant bacterial pathogens that contribute to both respiratory and wound infections. The results from these studies laid the foundation for use of gNO at a concentration higher than 160 ppm as an antibacterial agent, specifically for use against bacteria associated with clinical infections.

For this study, saline was selected as a suspension media because it would not mask the direct effect of gNO as a bactericidal, whereas fully supplemented growth medium might introduce external variables (e.g., buffer or react with gNO). Other media might also provide metabolites and replenish nutrients that produce enzymes that protect bacteria from oxidative and nitrosative damage, thereby masking the effect of gNO. Furthermore, it has been suggested that a saline environment better represents the hostile host environment that bacteria typically are exposed to in vivo. In saline, the colonies were static but remained viable. These conditions are similar to the approach previously used in animal models.

Table 1 present the results of this study of the effect of 200 ppm gNO on a variety of microbes.

TABLE 1

| | | Latent | | |
|---|---|---|---|---|
| | Gram | Period | −2.5 Log₁₀ | LD₁₀₀ |
| Bacteria | Staining | (hours) | (hours) | (hours) |
| S. aureus (ATCC) | Positive | 3 | 3.3 | 4 |
| P. aeruginosa (ATCC) | Negative | 1 | 2.1 | 3 |
| MRSA | Positive | 3 | 4.2 | 5 |
| Serracia sp. | Negative | 4 | 4.9 | 6 |
| S. aureus (Clinical) | Positive | 3 | 3.7 | 4 |
| Klebsiella sp.#1 | Negative | 3 | 3.5 | 6 |
| Klebsiella sp.#2 | Negative | 2 | 4.1 | 5 |
| Klebsiella sp.#3 | Negative | 3 | 5.1 | 6 |
| S. maltophilia | Negative | 2 | 2.8 | 4 |
| Enterobacter sp. | Negative | 4 | 5.3 | 6 |
| Acinetobacter sp. | Negative | 4 | 5 | 6 |
| E. Coli | Negative | 3 | 4.2 | 5 |
| Group B Streptococci | Positive | 1 | 1.5 | 2 |
| Mycobacterium | Positive | 7 | 9.2 | 10 |
| Average | | 2.77 | 3.82 | 4.77 |
| SD | | 1.01 | 1.17 | 1.3 |

As can be seen in Table 1, this study showed that gNO at 200 ppm had a complete bactericidal effect on all microorganisms tested. Without exception, every bacteria challenged with 200 ppm gNO had at least a 3 log₁₀ reduction in CFU/mL. Furthermore, every test resulted in a complete and total cell death of all bacteria. These results were characterized by a period of latency when it appeared that the bacteria were unaffected by gNO exposure. The latent period was then followed by an abrupt death of all cells; gram negative and gram positive bacteria, antibiotic resistant bacterial strains, yeast and mycobacteria all were susceptible to 200 ppm gNO. It is noted that the two drug resistant bacteria strains were also susceptible to treatment with gNO at 200 ppm.

These results indicate to a significant difference in the lag period for mycobacteria compared to all other organisms. The lag period suggests that mycobacteria may have a mechanism that protects the cell from the cytotoxicity of gNO for a longer period than other bacteria.

### Example 2

### Determination of Effective Antiviral Level of gNO

The efficacy of treating human influenza A with gNO has been studied. Two strains (H3N2 and H7N3) of the virus were studied and showed that treating influenza virions or incubated cells with 160 ppm exogenous gNO reduced not only viral replication but also their infectivity in a Madin-Darby Canine Kidney (MDCK) cell model of infection. gNO has been demonstrated as an effective anti-viral agent in both human Influenza A and highly pathogenic avian influenza.

The viruses used for the following experiments were from freezer stocks containing 1×10⁶-1×10⁷ pfu's/ml.

A standard plaque assay was used for the study. Frozen stock solutions of virions were diluted 1:10 in PBS and 3 ml were placed in each well of six well trays. The samples were either exposed to 160 ppm gNO or medical air at 37° C. Following exposure 0.5 ml was inoculated onto confluent MDCK cells, grown in six well trays, and incubated at 37° C. for 1 hour. The inoculums were then removed and 1:1 mixture of 2×DMEM and agar, with 2% trypsin, was added to each well and then incubated at 37° C. After 2 days the trays were fixed with 3.7% formaldehyde and the agar was removed from each well. The wells were then stained with crystal violet revealing the plaques.

A standard plaque assay was used for a hemagglutination assay. Frozen stocks of virions were diluted 1:10 in PBS and 3 ml were placed in each well of six well trays. The samples were either exposed to 160-20,000 ppm gNO or medical air at 37° C. For measure the effect of gNO on pH, when a large concentration of NO is added to saline the pH falls, therefore, a standard acid/base buffers were used to match the pH in the control to that of the treated. Following exposure the samples were diluted 1:2 in round bottom 96 well trays. Guinea pig red blood cells were added and agglutination was measured according to standard procedures.

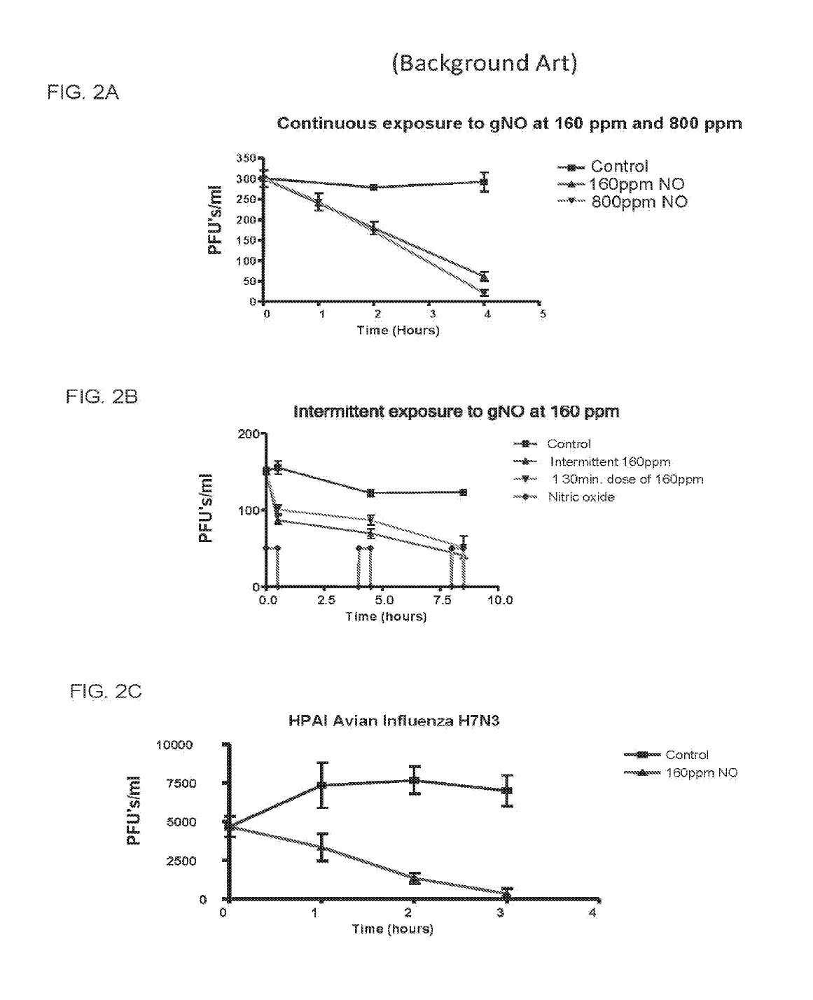

FIGS. 2A-C present plot of viral growth as a function of time measured for influenza A/victoria/H3N2 virions after exposure to nitric oxide 160 ppm and 800 ppm continuously for 4 hours (FIG. 2A), the same virions after being exposed to one gNO dose over 30 minute as compared to three 30 minute treatments Q4h (FIG. 2B), and the effect of continuous exposure to gNO at a concentration of 160 ppm for 3 hours of the highly pathogenic Avian Influenza H7N3.

As can be seen in FIGS. 2A-C, gNO has been shown as capable of reducing the infectivity of 2 strains of human influenza A/Victoria/H3N2 and HPAI/H7N2 viruses, and that the anti-viral effect of exposure to 160 ppm gNO is more evident in the intermittent form of exposure.

The efficacy of treating viral infection by respiratory syncytial virus by the methods presented herein was tested by exposure for 30 minutes of human respiratory syncytial virus (rgRSV30) to a gas mixture containing 160 ppm nitric oxide, using standard plaque assay as described hereinabove.

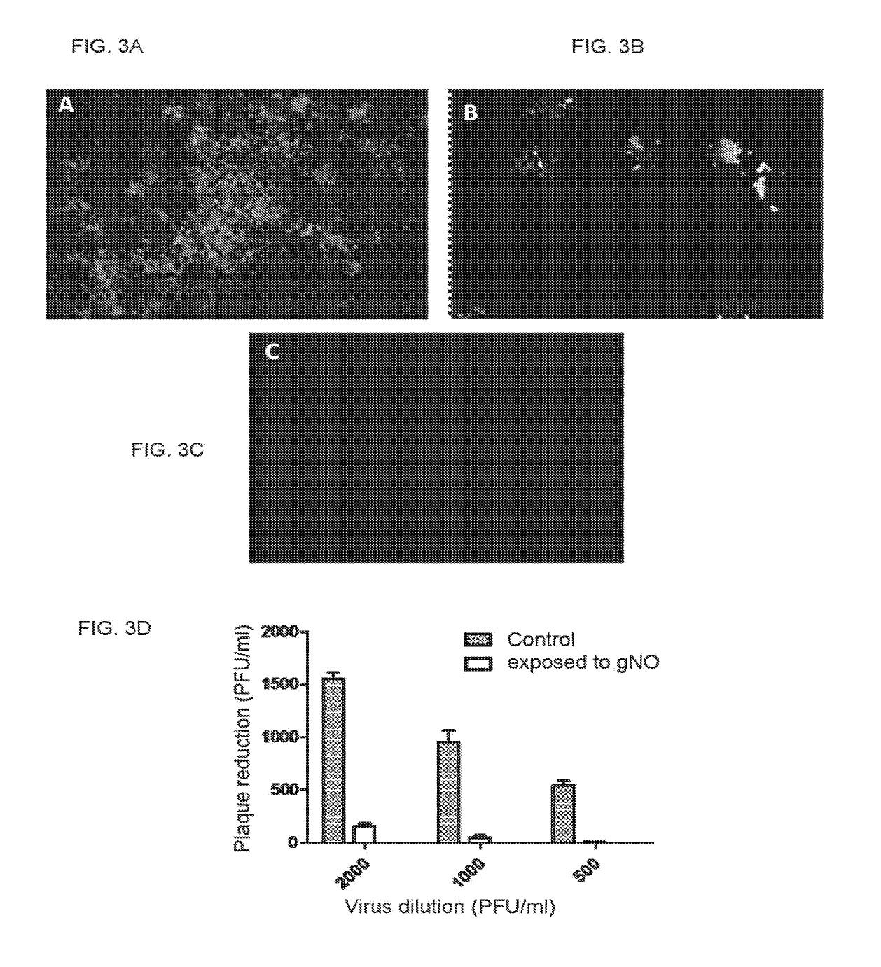

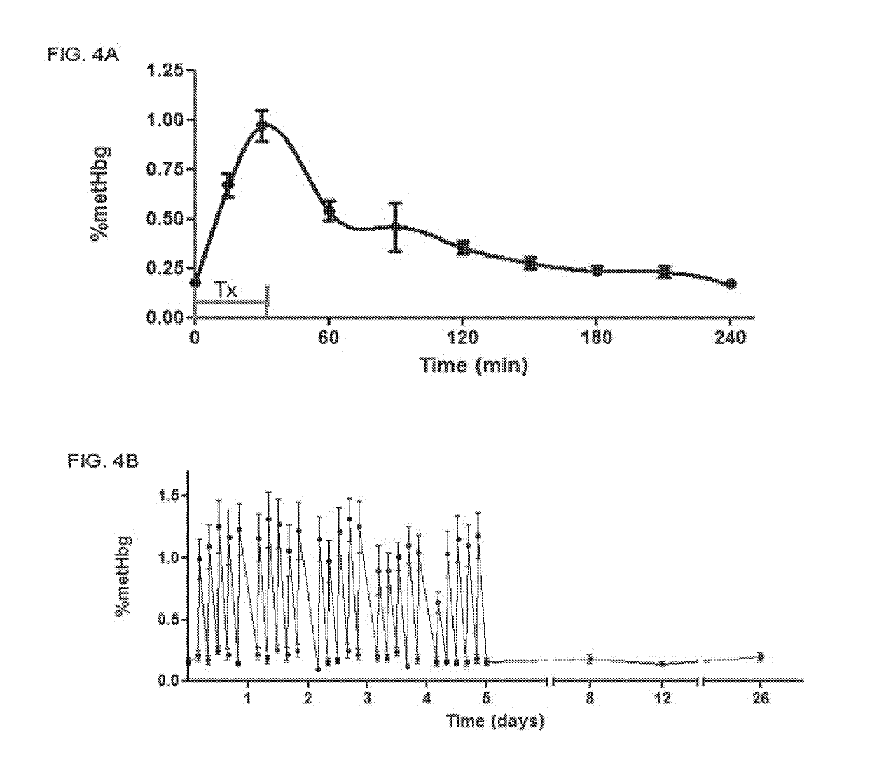

FIGS. 3A-D present the data obtained in the experiment using tissue culture samples harboring human rgRSV30, coupled to a green fluorescent protein, wherein the control experiment the samples were exposed to a ambient air (data not shown), and the tested samples having a starting viral level of 2000 PFU (FIG. 3A), 1000 PFU (FIG. 3B) and 500 PFU (FIG. 3C), were exposed to 160 ppm gNO for 30 minutes, whereas FIG. 3D presents a comparative bar plot comparing the control to test results.

As can be seen in FIGS. 3A-D, when the starting plaque-forming unit (PFU) of RSV was 2000 and 1000 PFU, a single exposure of 30 minutes to 160 ppm gNO reduced the virus viability by a factor of bout 10, and at a starting level of 500 PFU, viral viability was substantially nullified.

### Example 3

### Administration of gNO to Healthy Human Subjects

Cohort:

10 healthy adult volunteer subjects (5 males, 5 females), aged 20 to 62 years, were enrolled in the study after screening their medical history, a physical examination, pulmonary function tests and blood values. Exclusion criteria included individuals less than 19 years of age, pregnant females and unwilling to practice birth control during the study, diagnosed with pulmonary disease, epistaxis, hemoptysis, methemoglobinemia, organ transplant recipient or receiving antibiotic therapy.

Regimen and Post-Treatment:

After obtaining informed consent, treatment was initiated within 5 days of enrollment. Subjects were housed in a hospital ward and received 160 ppm gNO for 30 minutes every four hours (Q4h), five times a day, for five consecutive days by inhalation. Subjects returned for follow-up evaluations 3, 7 and 21 days after the final gNO administration. Subject safety was determined by monitoring vital signs, methemoglobin levels, lung function, blood chemistry, hematology, prothrombin time, inflammatory cytokine/chemokines levels and endothelial activation. These parameters were compared to baseline and at various time-points during and after gNO administration.

Device:

Subjects were administered gNO through a modified disposable mouthpiece to maximize mixing. Inspiration was spontaneously initiated by the subject from a conventional intermittent positive pressure breathing respirator (Mark-7, Carefusion, USA) in fixed flow mode delivering 48 liters per minute (LPM). Flows of gas were verified with a calibrated mass flow meter (TSI, USA). Gaseous nitric oxide (gNO, obtained at INOmax, Ikaria, USA) at a concentration of 800 ppm delivered at a flow of 12 LPM was titrated into a distal delivery port on the mouth piece connected to the respirator during inspiratory phase only (pressure switch). The Mark 7 respirator was supplied by an air/oxygen blender (Bird Sentry, Carefusion, USA) set to deliver 26% oxygen.

All components of the gNO delivery system were approved by the Therapeutic Product Directorate of Health Canada.

Monitoring of Chemicals and Physiological Parameters During Administration:

The levels of gNO, NO₂, O₂ and methemoglobin were monitored during the administration of gNO. The target gas mixture was 160 ppm gNO with a nitrogen dioxide (NO₂) level of less than 5 ppm and an oxygen (O₂) level ranging from 21% to 25%. Inspiratory NO, NO₂ and O₂ levels were continuously monitored by sampling from the mouthpiece sample port located about 6 millimeters from the mouth of the subject with an AeroNOx (Pulmonox, AB, Canada) NO, NO₂ and O₂ electrochemical analyzer. Delivery safety was determined by the number of occasions that NO₂ exceeded 5 ppm, gNO exceeding 10% variation and O₂ dropping below 20% during gNO administration. A commercially available noninvasive pulse oximeter (Rad 57, Masimo Corporation, USA) was used to measure saturation levels at the periphery of methemoglobin (SpMet).

These parameters were measured continuously during every gNO administration course and for 3.5 hours after the first treatment of the day. Daily serum samples were collected and frozen at −80° C. and the serum nitrite/nitrite level was measured using the Griess reagent.

Subjects underwent full pulmonary function tests (PFT), including lung diffusing capacity (DLCO) by a trained technician utilizing a calibrated pulmonary function system (Jaeger MasterScreen, VIASYS Healthcare, USA) on screening and days 2, 8, 12 and 26. Spirometry test (Microloop by Micro Medical, England) was performed on days 1, 3 and 4. Effect of gNO on lung function and DLCO was determined by changes from baseline, treatment days and follow up days.

General medical examinations were performed by a pulmonary physician on screening and on days 8, 12 and 26 to obtain oxygenation and vital sign measurements. Abbreviated physical examination by a registered nurse was carried out each day prior to initiation of treatments on days 1-5. Oxygenation was measured with a pulse oximeter (Rad 57, Masimo Corporation, USA) which was used according to manufacturer's guidelines to measure functional oxygen saturation of arterial hemoglobin (SpO₂) and heart rate. These parameters were measured continuously during every gNO administration and for 3.5 hours after the first treatment of the day. Cardiovascular status was determined by monitoring heart rate, blood pressure, respiratory rate and temperature. Values were recorded prior to the start of each gNO administration, following a 5 minute rest. During treatments, vital signs (except temperature) were also performed 15 minutes after the start of the treatment and at the end of gNO administration and recorded. After the first treatment each day, vital signs were recorded at every 30 minutes until the start of the second gNO administration of the day.

Hematological assessment included a complete blood count and differentials (hemoglobin, hematocrit, red blood cell count, white blood cell count, white blood cell differential, and platelet count) were obtained in order to monitor blood chemistry, hematology and inflammation measurements. The blood chemistry profile included serum creatinine, and liver function tests such as aspartate aminotransferase (AST) serum glutamic oxaloacetic transaminase (SGOT), alkaline phosphatase, and gamma-glutamyl transferase (GGT). The effect of gNO on coagulation was determined by the prothrombin time (PT) and its derived measures of prothrombin ratio (PR) and international normalized ratio (INR). Heparinized plasma was collected at baseline and on days 1, 2, 4, and 5 of gNO administration, and on follow-up days 3, 7 and 21 and frozen at −80° C. Plasma cytokine levels were assessed using the human inflammation cytokine bead array kit (BD Bioscience, Canada). Plasma levels of angiopoietin Ang-1 and Ang-2 were determined by ELISA (R&D Systems, USA).

A total of 750 measurements of gNO were recorded during the study. The average inspired gNO was 163.3 ppm (SD=4.0). The highest gNO concentration recorded was 177 ppm. The highest NO₂ level recorded during the treatments was 2.8 ppm (mean: 2.32; 95% confidence level: 2.17-2.47 ppm) and none of the subjects experienced a NO₂ level higher than 5 ppm. This was consistent with the performance specifications provided by the manufacturer of the apparatus of 1.56 ppm (SD=0.3). Of the 300 recorded oxygen values, the average oxygen level was 22.0% (SD=0.22%).

Data Analysis:

Descriptive statistical characteristics of the subjects prior to, during, and at the end of the study were tabulated and expressed as mean±standard deviation (SD). Differences in continuous variables (methemoglobin, serum nitrites/nitrates and SpO₂ levels) over the course of the study were analyzed utilizing repeated measures analysis of variance. Categorical events (number of subjects with a particular adverse event) were determined by constructing 95% confidence limits for their incidence. Differences between continuous variables at two specific times were evaluated with the paired t-test. Categorical events such as clinical pulmonary function and lung diffusion changes, changes in serum inflammatory markers, hematology, clinical chemistry and incidence of adverse events were analyzed by constructing 95% confidence limits for their incidence.

The data were analyzed using the unpaired Mann-Whitney test for comparison between any two groups and ANOVA for repeated measures of variance. Baseline comparisons were analyzed by repeated measures ANOVA with Bonferroni post test for parametric data, or Friedman test with Dunn's post test for non-parametric data.

Data analysis and graphical presentation were done using a commercial statistics package (Graphpad-Prism V 3.0, GraphPad Software Inc., USA).

Unless otherwise specified, p<0.05 indicated statistical significance. Results were represented by mean±SD from at least three independent measurements.

Results of Safety Studies:

Medical observation of adverse effects and general safety issues, concerning the repeated delivery of gNO at a concentration of 160 ppm into the airways of 10 healthy adult individuals, was effected by monitoring excessive NO₂ levels, while maintaining acceptable arterial hemoglobin oxygen saturation (SpO₂). A total of 250 gNO administration procedures were conducted to 10 subjects during the study period. All treatments were well tolerated and no significant adverse events were observed. Three minor adverse events were reported: One subject reported bruising of the arm from multiple attempts to successfully draw blood, while two other subjects reported a numbing sensation of the tongue during gNO administration. This was resolved by instructing the subject to relax and reposition the mouth piece.

During and after gNO administration, all vital signs remained within normal limits for age and with respect to baseline values. Specifically, there was no drop in blood pressure (which could potentially occur due to the vasodilator effect of gNO administration) during or after gNO administration. No sudden incidences of hypoxemia (less than 85% SpO₂) were observed during or after gNO administration. The lowest observed SpO₂ was 93%. SpO₂ levels over time decreased slightly between the pretreatment and post treatment but neither differed significantly statistically nor clinically. ANOVA analysis ruled out that this decrease was associated with the five repeated exposures to gNO over the course of the same day.

FIGS. 4A-B present results of monitoring methemoglobin levels before, during and after inhalation of 160 ppm of gaseous nitric oxide by 10 healthy human individuals, undergone 5 gNO administration courses daily, each lasting 30 minutes, for 5 consecutive days, while methemoglobin levels were measured using a pulse oximeter, wherein FIG. 4A is a plot of methemoglobin levels by percents as a function of time as measured before (time point 0), during 250 individual 30 minutes gNO administration courses (time interval of 0 to 30 minutes), after the courses (time interval of 30 to 60 minutes) and at 120 minutes, 180 minutes and 240 minutes after gNO administration was discontinued, and FIG. 4B is a plot of methemoglobin levels by percents as a function of time as measured at the beginning and end of 30 minutes gNO administration courses given over the course of 5 days, and followed 8, 12 and 26 days after gNO administration was discontinued.

As can be seen in FIG. 4A, all 930 recorded methemoglobin percent levels (SpMet) remained below the acceptable maximal level of 5%. The initial baseline SpMet was 0.16 (SD=0.10) percent. The highest SpMet was observed at the end of the 30 minutes treatment and was 2.5% with an average increase of 0.9% (SD=0.08). SpMet increased as predicted by about 1% between pretreatment and post treatment (p<0.001) and returned to baseline after 3.5 hours prior to the next gNO administration.

As can be seen in FIG. 4B, ANOVA analysis ruled out that this increase was associated with repeated treatments on the same day, as there was no accumulative or lingering effect on SpMet after five daily treatments for five consecutive days. Follow-up SpMet measurements on 3, 7 and 21 days after the final exposure to gNO on day 5 did not show any residual increase in SpMet.

Methemoglobin is reduced by an enzymatic reductase resulting theoretically in an increase in blood nitrite/nitrate levels. However, no significant differences in serum nitrite/nitrate levels from baseline were observed during the trial. One subject had significantly higher peak nitrite and nitrate values (p<0.001) which was also slightly different at baseline (p=0.038) compared to the other subjects.

There were no statistically, nor clinically significant changes in blood coagulation parameters, clinical chemistry and hematological parameters from baseline to completion of day 5. Although eosinophil cell numbers decreased during the study (baseline 0.15 giga/L; SD=0.12; end of study: 0.19 giga/L (SD=0.19), this difference was not significant (p=0.104). A 1% increase in neutrophil cell numbers from a baseline value of zero to 0.01 giga/L at the end of study was found, which also did not reach statistical nor clinical significance (p=0.169).

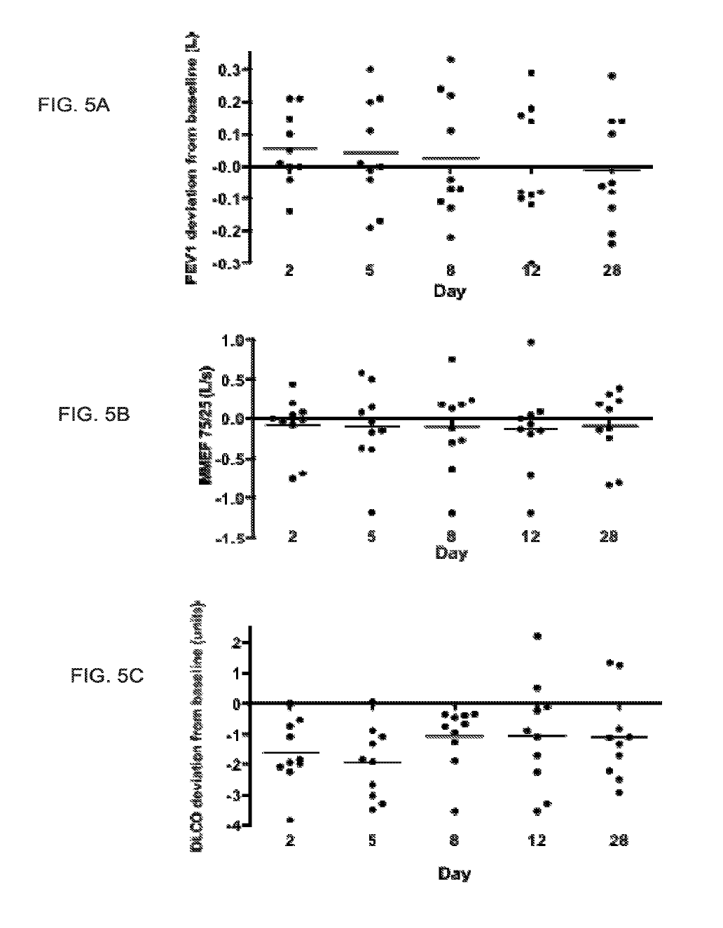

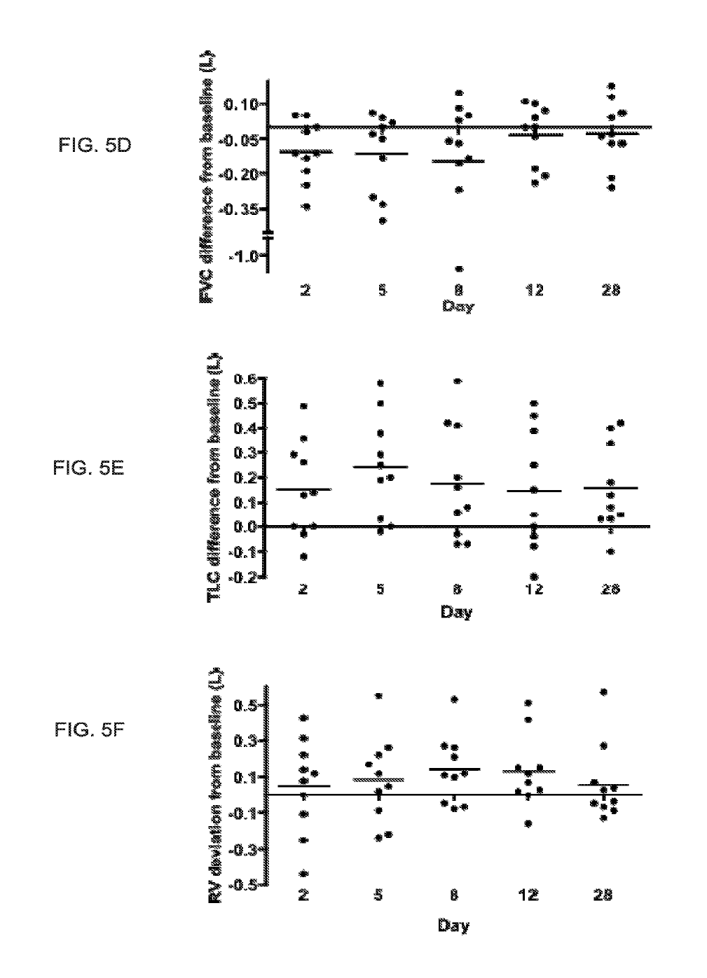

FIGS. 5A-F present various results of monitoring pulmonary function before, during and after inhalation of 160 ppm of gaseous nitric oxide by 10 healthy human individuals, wherein baseline values of pulmonary function tests were obtained within 7 days prior to gNO administration, and values during gNO administration were obtained on day 2 of the 5-days treatment and other data were obtained after the final gNO administration on day 5 and on days 8, 12 and 26, wherein FIG. 5A presents forced expiratory volume in 1 second in percents (FEV₁), FIG. 5B presents maximum mid-expiratory flow (MMEF), FIG. 5C presents carbon monoxide diffusing capacity (DLCO), FIG. 5D presents forced vital capacity (FVC), FIG. 5E presents total lung capacity (TLC) and FIG. 5F presents residual volume (RV), while all data are presented as means of all ten subjects and absolute differences compared to baseline prior to gNO administration, and statistical differences were assessed by Mann-Whitney test.

As can be seen in FIGS. 5A-F, pulmonary function tests did not reveal any abnormalities for any subjects during and after gNO administration treatments. Specifically, airflow as measured by FEV₁ and maximum mid-expiratory flow (MMEF) did not differ from baseline during the course of the study. Other lung function measurements such as DLCO, forced vital capacity (FVC), total lung capacity (TLC) and residual volume (RV) also did not change from baseline measurement.

To assess whether gNO inhalation may cause inflammation or endothelial activation cytokines and the vascular endothelium activation factors Ang-1 and Ang-2 were quantified in peripheral plasma at baseline at various time points thereafter.

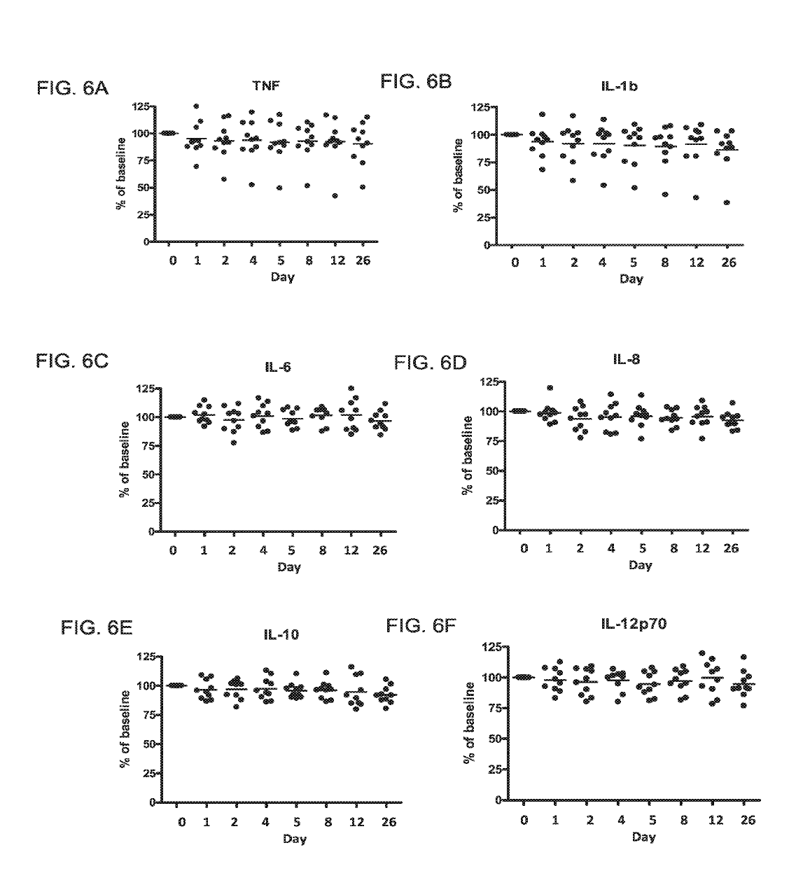

FIGS. 6A-F present blood levels of various cytokines before and after inhalation of 160 ppm gaseous nitric oxide by 10 healthy human individuals, as measured from blood samples collected within 7 days prior to gNO administration, each day during the treatment and 8, 12 and 26 days thereafter, wherein FIG. 6A presents the plasma levels of tumor necrosis factor (TNF)α, interleukin (IL)-1β data is presented in FIG. 6B, IL-6 in FIG. 6C, IL-8 in FIG. 6D, IL-10 in FIG. 6E and IL-12p70 in FIG. 6F, as determined by a cytometric bead array while statistical differences are compared by repeated measures ANOVA with Bonferroni post test for parametric data (IL-6, IL-8, IL-10, IL-12p70), or Friedman test with Dunn's post test for non-parametric data (TNF and IL-1b).

As can be seen in FIGS. 6A-F, cytokine levels of TNF, IL-6, IL-8, IL-10, IL-1b and IL-12p70 were unaffected by inhalation of gNO as compared to baseline. Comparisons between baseline cytokine levels and levels at each of the sampling time points for all 10 human participants resulted in no significant differences, compared by repeated measures ANOVA with Bonferroni post test for parametric data, or Friedman test with Dunn's post test for non-parametric data.

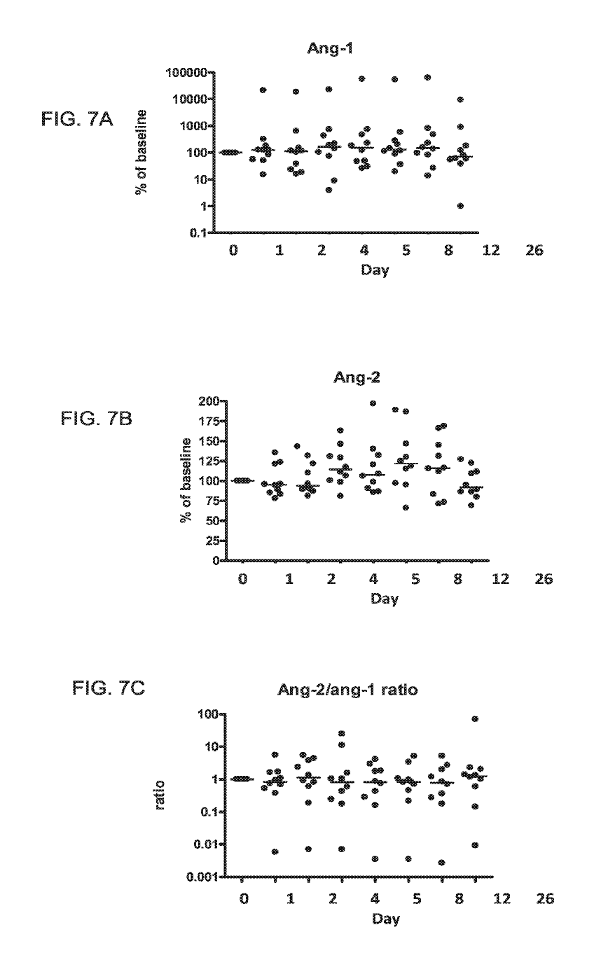

FIGS. 7A-C present plasma levels of angiopoietins Ang-1 and Ang-2 before and after inhalation of 160 ppm gaseous nitric oxide by 10 healthy human individuals, as measured in blood sample collected within 7 days prior to gNO inhalation, each day during gNO administration and 8, 12 and 26 days thereafter, wherein plasma levels of Ang 1 are shown in FIG. 7A, Ang-2 in FIG. 7B, and Ang-2/Ang-1 ratios in FIG. 7C, as determined by using a cytometric bead array while statistical differences were assessed compared by Friedman test with Dunn's post test.

As can be seen in FIGS. 7A-C, Ang-2 and Ang-2/Ang-1 ratios were not affected in this study. Outlier data in FIGS. 4A-C did not show any correlation with changes in any of the other parameters, and thus appears to be isolated findings of unknown significance.

### CONCLUSIONS

The safety of a treatment of human by inhalation of gNO at a concentration of 160 ppm, has been demonstrated and presented herein. It has been shown herein that 160 ppm gNO can be safely delivered to healthy human lungs in a pulsed manner for five consecutive days, showing no significant adverse events. All vital signs remained well within acceptable clinical margins during and several days after gNO administration at 160 ppm.

At least with regards to methemoglobin and NO₂ levels, the findings presented herein are superior to findings obtained for continuous inhalation of 80 ppm gNO, which is the currently approved gNO dose for inhalational use in full term infants, presumably due to the intermittent dosing strategy utilized herein. While continuous delivery of 80 ppm gNO has been reported to cause at least 5% increase of SpMet levels, with 35% of the subjects exceeding 7%, the results presented hereinabove (all 930 recorded SpMet levels) remained below 5%.

While the expected increase in methemoglobin levels during one treatment course was estimated at 1%, the observed average rise of 0.9% methemoglobin for the ten individuals in a single treatment course was consistent with first order pharmacokinetics model estimates, considering the ±1% absolute accuracy of the pulse oximeter. The study established that 3.5 hour interim period allowed the methemoglobin concentration to return to baseline, thereby allowing five daily cycles for five days without a significant clinical increase in methemoglobin concentrations. Taken together, it has been shown herein that intermittent gNO dosing strategy is safe for humans with regard of methemoglobin production and metabolic burden.

Similarly, the mean peak concentrations of NO₂ level shown hereinabove (2.8 ppm) is comparable with that observed during continuous delivery of 80 ppm (2.6 ppm) of previous studies. The limitations of this and other studies with regard to gNO delivery are that the NO and NO₂ levels are only known at the entry point into the subjects' respiratory tract and the actual resulting levels of oxides of nitrogen in the lung are unknown. Despite this resilience to nitrosative stress, it may well be prudent in future studies to screen subjects for thiol and methemoglobin reductase deficiencies.

The study presented hereinabove also demonstrates that 160 ppm of gNO, delivered as outlined, impacts lung function only minimally, and acute airway inflammation, measured by determining flow rates, was not detectable. Possibly, potential deleterious airway reactivity could be masked or prevented by the ameliorative smooth muscle relaxation that is known to be exerted by gNO. In patients with pulmonary infection, high NO delivery might cause an increase in airway reactivity. However, the vasodilatory activity of NO may benefit the patient in addition to the antimicrobial activity of NO.

The delivery of 160 ppm NO to humans shown herein did not cause lung parenchymal injury, as measured by different lung function parameters. Likewise, plasma inflammatory cytokine levels, the earliest host responses to lung injury, and levels of eosinophils and neutrophils remained constant during and days after gNO inhalation. In addition, the vascular endothelial activation factors Ang-1, Ang-2 and the Ang-2/Ang-1 ratio were unaffected by gNO administration by inhalation.

Pulmonary function mechanics and inflammatory markers remained unchanged compared to baseline values in measurements three days and 28 days post treatment by gNO administration. While it cannot be exclude that some longer term change may occur in lung function, the absence of any sign of inflammation in the post treatment period shown hereinabove makes this unlikely. If serum inflammatory markers may prove insensitive to measure acute or even chronic changes in the lungs, inflammatory markers from bronchoalveolar lavage (BAL) fluids could be sampled.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims.

All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention. To the extent that section headings are used, they should not be construed as necessarily limiting.

What is claimed is:

1. A method of treating a human subject suffering from a disease or disorder that is manifested in the respiratory tract or a disease or disorder that can be treated via the respiratory tract, said disease or disorder being associated with a nosocomial infection, the method comprising subjecting the subject to intermittent inhalation of gNO at a concentration of at least 160 ppm, thereby treating the disease or disorder.

2. A method of treating a human subject prone to suffer from, or being at risk of suffering from, a disease or disorder that is manifested in the respiratory tract or a disease or disorder that can be treated via the respiratory tract, said disease or disorder being associated with a nosocomial infection, the method comprising subjecting the subject to intermittent inhalation of gNO at a concentration of at least 160 ppm, thereby treating or preventing the disease or disorder.

3. The method of claim 2, wherein said human subject is prone to suffer said disease or disorder due to general, environmental and occupational conditions.

4. The method of claim 3, wherein said human subject is selected from the group consisting of elderly people, medical staff and personnel (doctors, nurses, caretakers and the likes) of medical facilities and other care-giving homes and long-term facilities, commercial airline crew and personnel (pilots, flight attendants and the likes), livestock farmers and the likes.

5. The method of claim 1, wherein said nosocomial infection is an infection stemming from direct-contact transmission, indirect-contact transmission, droplet transmission, airborne transmission, common vehicle transmission and vector borne transmission.

6. The method of claim 1, wherein said nosocomial infections is caused by an antibiotic resistant bacterium.

7. The method of claim 6, wherein said bacterium is selected from the group consisting of carbapenem-resistant Klebsiella (KPC) or other Enterobacteriaceae, methicillin resistance Staphylococcus Aureus (MRSA), Group A Streptococcus, Staphylococcus aureus (methicillin sensitive or resistance), Neisseria meningitides of any serotype and the likes.

8. A method of treating a human subject suffering from a disease or disorder that is manifested in the respiratory tract or a disease or disorder that can be treated via the respiratory tract, said disease or disorder being an opportunistic infection in an immuno-compromised subject, the method comprising subjecting the subject to intermittent inhalation of gNO at a concentration of at least 160 ppm, thereby treating the disease or disorder.

9. (canceled)

10. The method of claim 1, further comprising monitoring, during and following said subjecting, at least one on-site parameter selected from the group consisting of:
a methemoglobin level (SpMet);
an oxygen saturation level (SpO₂);
an end tidal CO₂ level (ETCO₂); and
a fraction of inspired oxygen level (FiO₂),
and/or at least one off-site parameter selected from the group consisting of:
a serum nitrite level (NO₂⁻); and
an inflammatory cytokine plasma level,
in the subject.

11. The method of claim 10, comprising monitoring at least two of said parameters.

12. The method of claim 10, comprising monitoring all of said parameters.

13. The method of claim 10, wherein a change in said at least one of said parameters following said subjecting is less than 2 acceptable deviation units from a baseline.

14. The method of claim 11, wherein a change in at least two of said parameters following said subjecting is less than 2 acceptable deviation units from a baseline.

15. The method of claim 12, wherein a change in all of said parameters following said subjecting is less than 2 acceptable deviation units from a baseline.

16. The method of claim 10, wherein a change in at least one of said on-site parameters following said subjecting is less than 2 acceptable deviation units from a baseline.

17. The method of claim 10, wherein a change in at least one of said off-site parameters following said subjecting is less than 2 acceptable deviation units from a baseline.

18. The method of claim 1, further comprising monitoring urine nitrite level in the subject.

19. The method of claim 18, wherein a change in said urine nitrite level following said subjecting is less than 2 acceptable deviation units from a baseline.

20. The method of claim 1, further comprising monitoring in the subject at least one off-site parameter selected from the group consisting of: a hematological marker;
a vascular endothelial activation factor;
a coagulation parameter;
a serum creatinine level; and
a liver function marker, in the subject.

21. The method of claim 20, wherein a change in at least one of said off-site parameters following said subjecting is less than 2 acceptable deviation units from a baseline.

22.-33. (canceled)
